(19) United States
(12) Patent Application Publication
(10) Pub. No.: US 2020/0281688 A1
(43) Pub. Date: Sep. 10, 2020

(54) DENTAL HANDPIECE, MOTOR AND COUPLER WITH MULTI-WAVELENGTH LIGHT OUTPUTS

(71) Applicant: LARES RESEARCH, Chico, CA (US)

(72) Inventors: Craig Lares, Chico, CA (US); Jason Orgain, Chico, CA (US); Bruce Holderbein, Chico, CA (US)

(73) Assignee: LARES RESEARCH, Chico, CA (US)

(21) Appl. No.: 16/816,708

(22) Filed: Mar. 12, 2020

Related U.S. Application Data
(63) Continuation of application No. PCT/US2018/050917, filed on Sep. 13, 2018.
(60) Provisional application No. 62/695,905, filed on Jul. 10, 2018.
(60) Provisional application No. 62/630,090, filed on Feb. 13, 2018.
(60) Provisional application No. 62/558,152, filed on Sep. 13, 2017.

(51) Int. Cl. A61C 1/08; A61B 1/24; A61B 1/06; A61C 1/05; A61C 1/12; A61C 3/02; A61C 17/18; A61B 1/07; H02K 7/14; H02K 11/00; H02K 11/28

(52) U.S. Cl. CPC A61C 1/088; A61B 1/24; A61B 1/0638; A61B 1/0684; A61C 1/052; H02K 11/28; A61C 3/02; A61C 17/18; A61B 1/07; H02K 7/145; H02K 11/0094; A61C 1/12

(57) ABSTRACT

Dental handpieces, motors, couplers and other dental instruments and methods having multiple very small, high output, LEDs in a single lamp assembly to facilitate incorporation of a plurality of wavelengths emitted from these devices to allow dentists to select between the benefits of multiple wavelength light outputs to the working area in a single device.

### CROSS-REFERENCE TO RELATED APPLICATIONS

This application claims priority to, and is a 35 U.S.C. § 111(a) continuation of, PCT international application number PCT/US2018/050917 filed on Sep. 13, 2018, incorporated herein by reference in its entirety, which claims priority to, and the benefit of, U.S. provisional patent application Ser. No. 62/695,905 filed on Jul. 10, 2018, U.S. provisional patent application Ser. No. 62/630,090 filed on Feb. 13, 2018, and U.S. provisional patent application Ser. No. 62/558,152 filed on Sep. 13, 2017. Priority is claimed to each of the foregoing applications. Each of the foregoing applications is incorporated herein by reference in its entirety.

The above-referenced PCT international application was published as PCT International Publication No. WO 2019/055678 A1 on Mar. 21, 2019, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to dental devices, and more particularly to dental devices with dual-mode illumination.

### 2. Background Discussion

Modern dental handpieces are designed to be as small and light as possible to minimize dentist hand fatigue and maximize oral cavity accessibility. The extremely limited space available in current day dental handpieces, motors and swivel couplers with industry standard connections makes it difficult to incorporate multiple LED lamps of a single wavelength output into these devices while maintaining small size and industry standard connectivity.

### BRIEF SUMMARY

Aspects of the present disclosure are devices and methods for combining multiple very small, high output, LEDs in a single lamp assembly to facilitate incorporation of more than one wavelength into these devices. Combining multiple wavelength LEDs in a single lamp as described herein further simplifies enabling the user to select between wavelengths and reduces the overall cost of the product.

This disclosure also details dental handpieces, motors, couplers and other dental instruments that allow dentists to select between the benefits of multiple wavelength light outputs to the working area in a single device. For example, white light emitted near the tip of the cutting instrument (bur) of a dental handpiece allows the dentist to see better due to increased white light aimed at the area of treatment. As a further example, with the help of bandpass filtered glasses, loupe inserts, etc., worn by the user, 405 nm light causes tooth decay to fluoresce orange/red, thereby allowing for easy detection of remaining caries during removal and also allows easy visualization of the margin between tooth colored restorative materials and tooth. During a given dental procedure, the dentist might need to use more than one wavelength and it is preferred that he/she be able to conveniently change among the available wavelengths to minimize the number of instruments that must be used and to limit the use of valuable chair time.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 illustrates an embodiment of a dual wavelength light emitting air driven dental handpiece swivel coupler according to the technology presented herein.

FIG. 2 illustrates an embodiment of a dual wavelength light emitting dental motor and motor control box according to the technology presented herein.

FIG. 3 illustrates an embodiment of a dual wavelength LED lamp for use in dental instruments according to the technology presented herein.

FIG. 4A shows a schematic diagram of a circuit diagram of the LED lamp of FIG. 3 in a first switch orientation.

FIG. 4B shows a schematic diagram of a circuit diagram of the LED lamp of FIG. 3 in a second switch orientation.

FIG. 5 shows a schematic diagram of a dual wavelength LED lamp optical barrier according to the technology presented herein.

FIG. 6 illustrates an embodiment of a dual wavelength light emitting air driven dental handpiece or mechanically driven contra angle according to the technology presented herein.

FIG. 7 illustrates an embodiment of a dual wavelength light emitting scalar for removing material from teeth according to the technology presented herein.

FIG. 8 illustrates an embodiment of a dual wavelength light emitting scalar for removing material from teeth according to the technology presented herein.

FIG. 9 shows a schematic diagram of an air driven or electric dental handpiece motor having multiple lamps or LED's that can be moved to alter illumination between wavelengths.

FIG. 10 shows a schematic diagram of an air driven or electric dental handpiece motor having a single lamp multi LED with a shutter.

FIG. 11 shows a schematic diagram of an air driven or electric dental handpiece motor having a single lamp with single white LED with a bandpass filter.

FIG. 12 shows a schematic diagram of a dental assembly comprising an electric-driven dental handpiece and handpiece motor in accordance with the present description.

FIG. 13 shows a perspective view of a dental handpiece swivel coupler having a single LED with dual wavelength output.

FIG. 14A shows a rear view of an electric dental handpiece motor having a bifurcated fiber optic element.

FIG. 14B shows a closeup view of an input channel of the fiber optic element of FIG. 14A.

FIG. 14C shows a frontal view of an electric motor having an orientation of LEDs or lamps so as to direct light into both channels of the handpiece of FIG. 14A.

### DETAILED DESCRIPTION

The systems and methods detailed below are directed to dental instruments that provide the ability to illuminate teeth surfaces in a plurality of wavelengths using the same device. One particularly beneficial method utilizes one or more of the devices details below for restoring a tooth utilizing a dental handpiece capable of emitting dual wavelength light whereby 405 nm light emitted from the dental handpiece is first used to detect and remove decay, restorative material(s) are then applied to the tooth, a 405 nm light is used to enhance visual differentiation between excess restorative material(s) and tooth while the excess restorative material(s) are removed, the wavelength emitted from the dental handpiece is changed from 405 nm to white light, and the white light is used to assist in the occlusal adjustment and final surface finishing of the restoration.

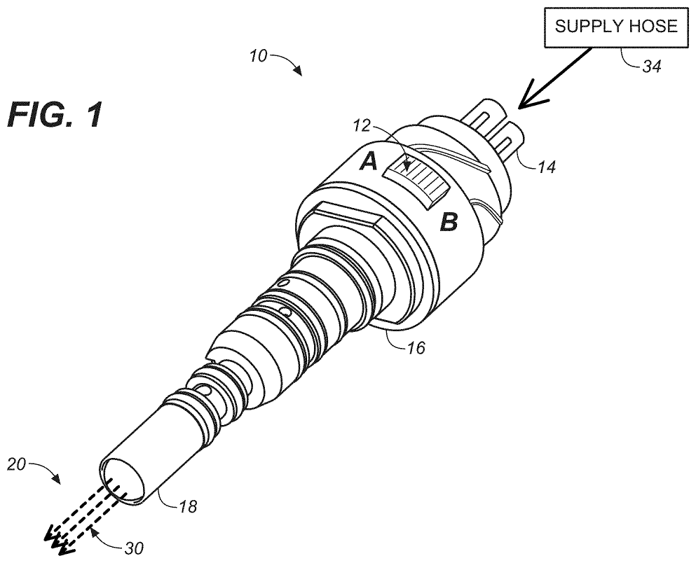

FIG. 1 shows a perspective view of an embodiment of an air driven dental handpiece swivel coupler 10 in accordance with the present description. Swivel coupler 10 comprises a first end 18 configured to releasably couple to an air driven dental handpiece (e.g. contra angle similar dental instrument), and a second end 14 configured to connect to an air/water/electricity supply hose 34. In a preferred embodiment, both ends 14 and 18 may comprise releasable or detachable connections.

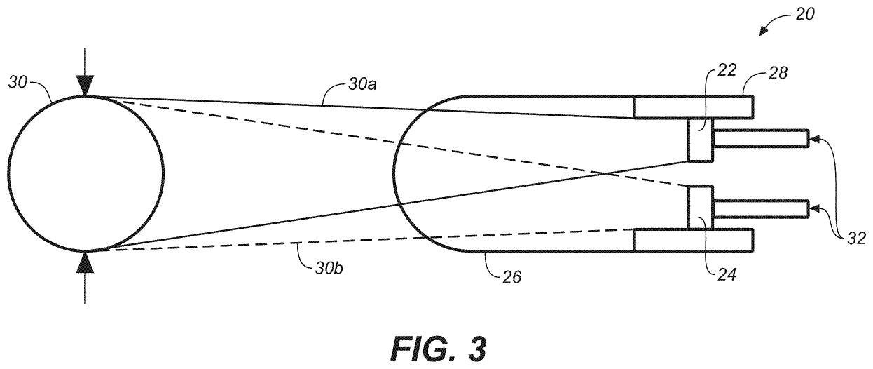

The handpiece end 18 of the swivel coupler 10 comprises an LED lamp 20 containing two LEDs of differing wavelengths (e.g., white and 405 nm, see FIG. 3). The lamp 20 may be fixed, or removable by the user. In one embodiment, the coupler lamp 20 is configured to emit light 30 from each wavelength LED into a transmitting element such as a fiber optic element in the center of the handpiece back end (both not shown) when the handpiece is attached to the coupler 10.

Coupler 10 generally acts as a conduit for transmitting electrical power, air or water from supply hose 34 for driving radial or other motion of the dental device when connecter to coupler 10. Electrical power may be supplied to the swivel coupler 10 by the supply hose 34 or by an air driven generator (not shown) internal or external to the swivel coupler 10, the air provided by the supply hose 34 to the coupler 10 or returning from the handpiece.

In a preferred embodiment, the coupler 10 includes a switching means or like mechanism for user selection of wavelength such as an electrical switch 12 and/or other components and circuitry as described herein. The coupler 10 may include identifying markings, e.g. letters or colors on its exterior to assist the user in identifying the desired position to change from a first wavelength to the second wavelength. In one embodiment shown in FIG. 1, the markings do not indicate wavelength (e.g. blue or white), but only A or B wavelength, since polarity may not be known until installation. In one embodiment, the markings are one or more dots, figures or other markings that are the color of the selected wavelength (e.g., white for illumination and blue for 405 nm). The markings can also be symbolic representations of the application (e.g., excitation, illumination, etc.) specific to the wavelength.

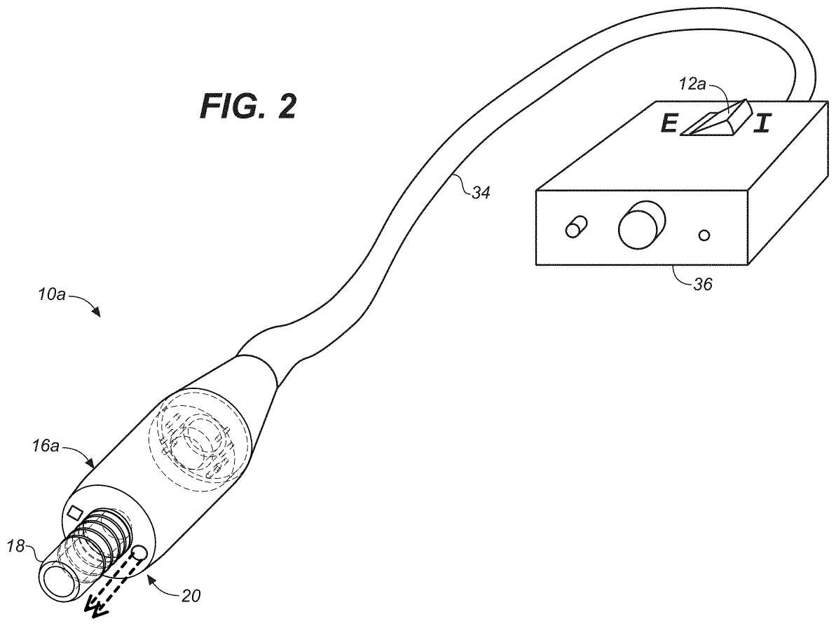

FIG. 2 shows an embodiment of an electric dental handpiece motor 10a and motor control box 36 according to the present description. The motor 10a is configured such that one end 18 of the motor body 16a connects to a mechanically driven contra angle (not shown) with an off-center fiber optic element and the other end connects to an air/water/electricity supply hose 34 that in turn connects to a motor control box 36, both of which preferably comprise detachable connections.

In one embodiment, an LED lamp 20 comprising two LEDs 22/24 of differing wavelengths (white and 405 nm, see FIG. 3) is disposed in/on the face of the motor body 16a that opposes the contra angle when it is connected. In the embodiment shown in FIG. 2, the lamp 20 is mounted off center and in alignment with a transmitting element such as a fiber optic element (not shown) in the contra angle when the contra angle is attached. In one embodiment, the motor 10 sends an illumination beam 30 (see FIG. 3) from each wavelength energized LED to enter the fiber optic element in the back end (proximal end) of the contra angle when the contra angle is attached to the motor 10a.

The motor control box 36 may include means for user selection of wavelength such as an exterior mounted electrical switch 12a. Identifying markings or colors may be included on the motor control box 36 exterior in proximity to the switch 12a to assist the user in actuating the switch to the correct position for the desired white or 405 nm wavelength

FIG. 3 shows a preferred embodiment of a dual-wavelength (white and 405 nm) LED lamp 20 for use in dental instruments, such as an air driven handpiece, handpiece coupler, handpiece air driven motor, handpiece electric motor, or handpiece contra angle, powered or manual scaler, or other dental instrument. LEDs of two different wavelengths, e.g. 405 nm LED 22 and white LED 24, are provided. It is appreciated that one or more LEDs may be employed for each wavelength, and other wavelengths may be employed other than white/405 nm.

In one embodiment, all LEDs 22/24 are oriented in the lamp 20 so as to emit their light output 30 (individually 30a and 30b respectively) in substantially the same direction and towards the same target. The LEDs 22/24 are preferably small enough, arranged in sufficient density, have sufficient output intensity, and are arranged in a manner that allows them to emit enough light at a diameter not to exceed 0.125 inches so as to accomplish the intended purpose of that wavelength (excitation, illumination, etc.).

In one embodiment, two electrical connections 32 on the lamp apply electrical current to one or more LEDs of one wavelength wired in parallel to all the LEDs of the second wavelength.

The LEDs 22/24 may be mounted in the lamp 20 in various ways.

For example, they are mounted at the end of two or more conducting pins 32 housed in a non-conducting material or housing 28. In another embodiment, there are two or more LEDs 22/24 mounted on each conducting pin 32 so as to effectuate a parallel circuitry between LEDs of different wavelengths. In another embodiment, LEDs 22/24 are mounted on a circuit board (not shown) sufficiently small to fit into the dental instrument with LEDs of different wavelengths wired in a parallel circuitry. As shown in FIG. 3, LEDs 22/24 are housed in a bulb 26.

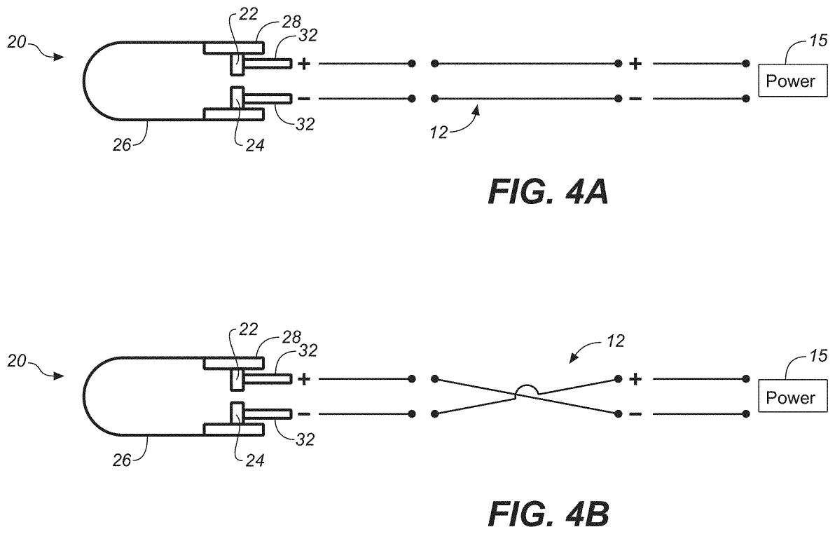

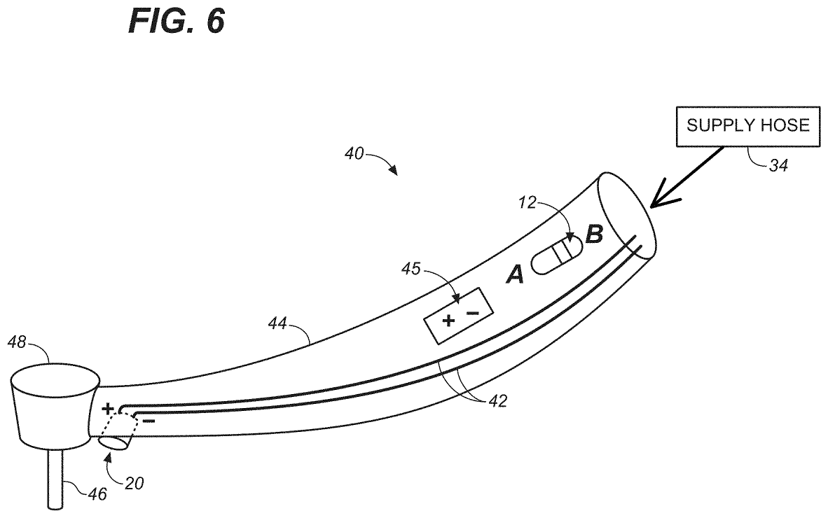

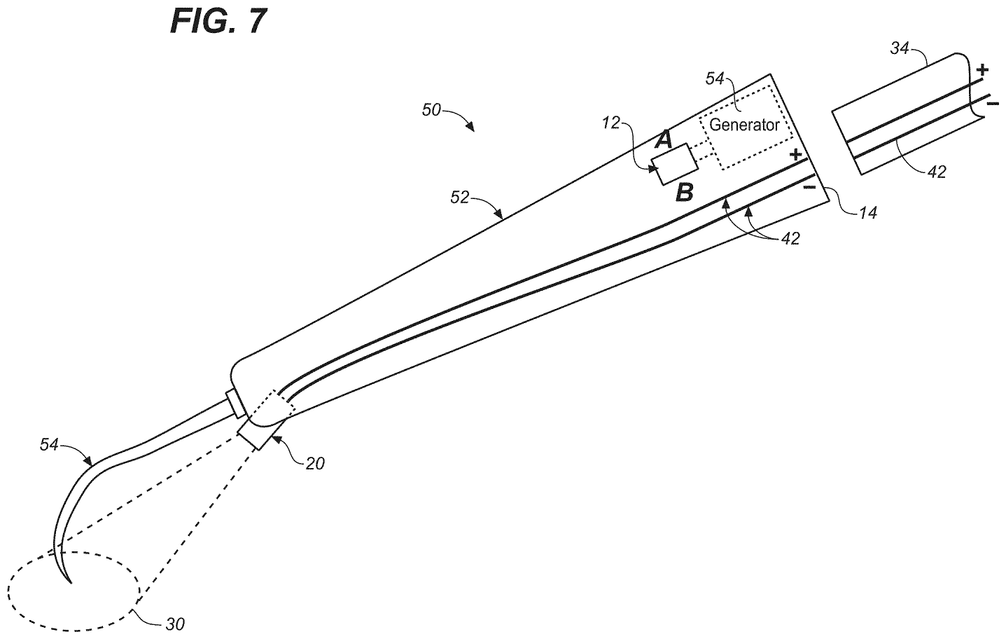

FIG. 4A and FIG. 4B show a schematic diagram of a circuit diagram of the LED lamp 20 of FIG. 3 and switch 12 in a first switch orientation and second switch orientation, respectively. In a preferred embodiment, switch 12 comprises a circuit arrangement and switching mechanism configured to effectuate a reversing of polarity, to thereby change from a first wavelength to a second wavelength. Switch 12 may be located in a variety of locations, e.g. swivel coupler 10 of FIG. 1, handpiece/contra angle (FIG. 6), scaler (FIG. 7), supply hose 34, motor 10a or on the motor control box 36 as switch 12a in FIG. 2 to effectuate a reversing of polarity, thereby changing from a first wavelength to a second wavelength. As shown in FIG. 4A and FIG. 4B, the conducting pins 32 of the LEDs 22/24 are wired to the power source 15 (from supply hose 34, motor control box 36, or other location) with the intervening switch 12 such that in first position A (FIG. 4A) power is delivered to the excitation (405 nm) light 22 (break in the circuit for white wavelength LED 24 and close the circuit for 405 nm LED 22), and in the second position B (FIG. 4B) the polarity is reversed so that power is delivered to the illumination (white) light 24 (break in the circuit for 405 nm wavelength LED 22 and close the circuit for white LED 24). The switch 12 may be manually actuated by the user without risk of loosening the supply hose 34 on the coupler 10 in various forms, such as a lever, button, rocker, slider or other small switching mechanism known in the art.

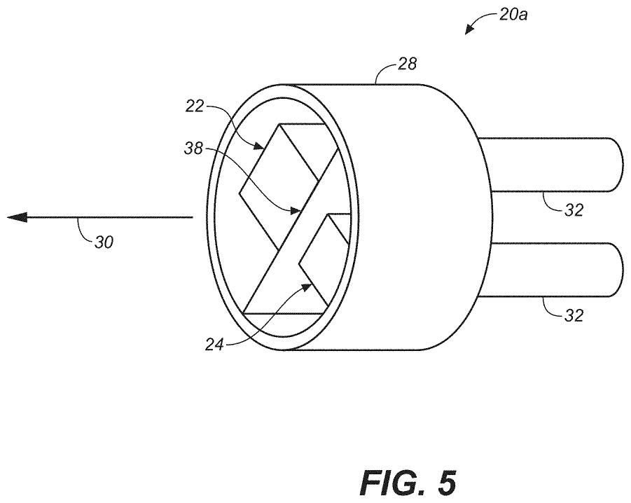

When the LEDs of one wavelength are powered, they may emit sufficient radiation so as to cause the adjacent LEDs of the non-powered wavelength to fluoresce and also emit radiation at the non-selected wavelength, thereby eliminating or diminishing the desired effect of the selected wavelength. FIG. 5 illustrates a perspective view of an alternative LED lamp 20a comprising a physical non-light transmitting optical barrier 38 disposed between the white 24 and 405 nm 22 LEDs to shield the non-powered LED from radiation emitted by the adjacent LED. In this configuration, fluorescence of non-powered LED(s) would be minimized or inhibited, thus minimizing or inhibiting emission of any non-selected wavelength along with the selected wavelength of emitted light 30.

In one configuration, the optical barrier 38 may be made of a non-conducting material such as epoxy. In another configuration, the optical barrier may be a non-light transmitting coating applied to all or a portion of the external surface(s) of one or both wavelength LEDs 22/24. The optical barrier 38 may be flat, curved or fully or partially cylindrical.

FIG. 6 shows an air driven dental handpiece or mechanically driven contra angle 40 in accordance with the present description. The proximal end of hand piece/contra angle 40 detachably connects to supply hose 34 for delivering air/water, power or mechanical coupling from a motor drive shaft (not shown) to power operation of a working instrument 46 (e.g. bur, etc.) located at distal end 48 of body 44. Depending on the configuration, electrical power may or may not be delivered from the supply hose 34.

A dual wavelength (white and 405 nm) LED lamp 20 is disposed at distal end 48 in close proximity to the working instrument 46. Electrical power transmission means 42 (such as electrical wiring) may be directed along the length of the body from the supply hose 34 coupling to power to the LED lamp 20. In one embodiment, when the handpiece/contra angle 40 is activated, one wavelength light is emitted via lamp 20 near or around the area of the working instrument 46 contact.

In one embodiment, the handpiece/contra angle includes means for user selection of wavelength such as an exterior mounted electrical switch 12. In an alternative embodiment, power to the lamp 20 may be supplied by the electrical generator 45 (e.g. fluid driven or mechanically driven) in handpiece/contra angle 40. In a further alternative embodiment, the lamp 20 may be powered from the air/water/electrical supply hose 34. Connection may be affected as a static separable connection, or as a rotating dynamic connection that swivels and is separable.

FIG. 7 shows an embodiment of a scaler 50 or other powered dental instrument for removing plaque, calculus or tartar from teeth. The instrument 50 includes a working tip 54 on the distal end of the instrument that comes in contact with the tooth for the removal of plaque, etc.

In various embodiments, one or more dual-wavelength lamps 20 (e.g. white and 405 nm LEDs) are arranged on the distal end of the instrument body 52 so as to generate a light beam 30 to illuminate the area of the tooth surrounding the working tip 54. The illumination can be used to aid in the visualization of remaining plaque, etc. and/or to direct the working tip 54 to areas of the tooth with remaining plaque, etc.

In one embodiment, electrical power may be supplied to the instrument 50 and lamp 20 by electrical leads 42 in the supply hose 34 releasably coupled at the proximal end 14 of the instrument 50, that run through the instrument body 52. A switch 12 may be included on the instrument body 52 or other location allowing the user to change wavelengths of the illuminated light 30.

In an alternative embodiment, the supply hose 34 may provide air and water that is delivered to proximal end 14 of the instrument 50 to power a fluid activated generator 54, which then provides power to the lamp 20 via electrical leads 42.

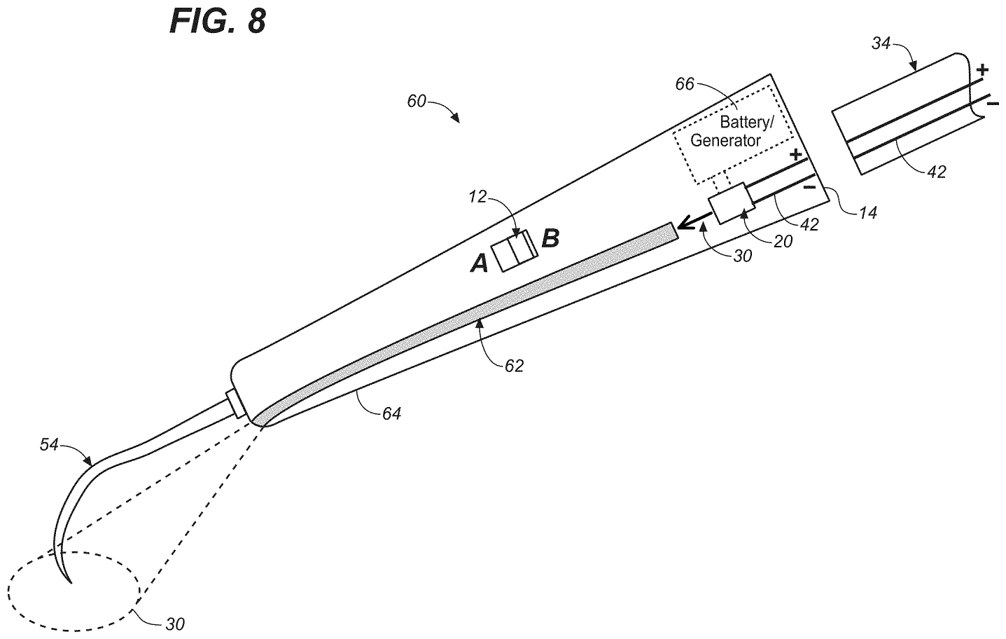

FIG. 8 shows another embodiment of a scaler 60 or other powered dental instrument for removing plaque, calculus or tartar from teeth, wherein the lamp 20 is located at proximal end 14 of the scaler body 64 (or in supply hose 34), and the illumination beam 30 is carried to the distal working end via a light guide 62, such as a fiber optic element or the like. In one embodiment, supply hose 34 provides electrical power via electrical leads 42 to the proximal end 14 of the scaler 60 to power lamp 20. Switch 12 may be disposed within housing 64 and coupled to lamp 20 or leads 42 connected thereto to allow manual switching between wavelengths (e.g. white and 405 nm light).

In an alternative embodiment, scaler 60 may comprise a battery or generator 66 that is used to power lamp 20. In the configuration using a generator 66, supply hose 34 may comprise air or water delivery lines for powering generator 66, which is then converted to electrical energy for use with lamp 20.

In a further alternative embodiment (not shown), lamp 20 may be disposed in the distal end of supply hose 34 such that illumination 30 is directed out of the distal end, and the light guide 60 extends to the proximal end 14 of body 64 to receive said illumination 30 upon coupling the supply hose 34 to the proximal end 14.

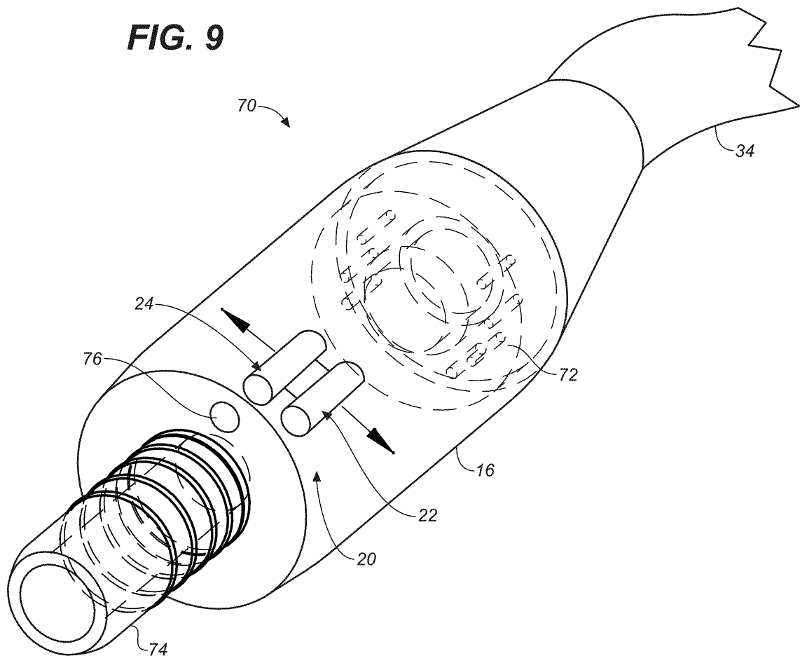

FIG. 9 shows a schematic diagram of an air-driven or electric dental handpiece motor 70 having multiple lamps 20 with single LEDs or different LED's of a multi-LED lamp, each being spaced at different radial, axial or circumferential distances from a centerline of internal motor 72. A centrally located shaft at the distal end 74 of motor 70 detachably connects to an attachable device such as a contra angle comprising a wave guide such as a fiber optic element (both not shown), while the proximal end of the motor body 16 detachably connects to an air/water/electricity supply hose 34.

Two or more single wavelength LED lamps 20 of differing wavelengths are located near the face of the motor and aperture 76 that opposes the contra angle or other dental instrument when it is connected at distal end 74. The first LED lamp or LED (e.g. white LED lamp 24) is initially located in substantial alignment with the aperture 76 (which in turn is aligned with fiber optic element in the contra angle when the contra angle is attached) allowing its light output to be efficiently transmitted into and through the fiber optic element of the contra angle. As seen in the embodiments shown in FIG. 9 through FIG. 11, the aperture 76 is off axis from the centrally located shaft at distal end 74. The lamps 20 are configured to be selectively moved into different locations via translational or rotational motion that may be linear, circular elliptical, or any other type of movement that serves to reposition the lamps 20 as needed relative to the aperture 76 and contra angle fiber optic element.

The motor 70 will also include means for user selection/motion of

LEDs and therefore wavelength. In one embodiment, each LED lamp 20 is mounted on a single shared element substantially flush with the face of the motor that is movable in order to reposition the lamps. Moving said element serves to reposition the LED's so that the first LED lamp in alignment with the aperture 76 and the contra angle fiber optic element is moved out of alignment, and a second or third LED lamp is moved into alignment thereby changing the wavelength. Further movement of the single shared element may be affected to deploy additional wavelength LED lamps.

In one embodiment, the contra angle may be removed by the user in order to access and move the shared element to effect wavelength change. Identifying markings or colors on the motor face and/or shared element may be used by the user to easily move the shared element to the right position for a desired wavelength.

Alternatively, the external surface of the motor body 16 may contain a lever, dial, ring or other mechanism (not shown) allowing the user to select a specific LED lamp and wavelength without removing the contra angle. Identifying markings or colors on the motor exterior and/or shared element may be used by the user to easily move the shared element to the right position for a desired wavelength. The lever, dial, ring or other mechanism may be oriented and coupled to the lamp(s) 20 to affect a translation, or rotation parallel or perpendicular to the motor 70 axis.

Each LED 22/24 mounted to the shared element shall have an electrical connection (e.g. electrical leads, not shown) to the motor electrical supply (e.g. from supply hose 34). The electrical connection may be continuous, and all LED's mounted to the shared element may be powered simultaneously, but only the LED in alignment with the contra angle fiber optic element will transmit light ultimately through the contra angle to the work area.

Alternatively, the electrical connection to each individual LED 22/24 mounted on the shared element may be dynamic such that when a specific LED 22/24 is moved into position in alignment with the contra angle fiber optic element the LED 22/24 changes from unpowered to powered by virtue of its change in position, and the other LED's are unpowered.

In another alternative embodiment, the electrical connection to each individual LED 22/24 mounted on the shared element may be switchable such that when a specific LED 22/24 is moved into position in alignment with the contra angle fiber optic element the LED 22/24 changes from unpowered to powered by virtue of its change in position, and the other LED's are unpowered.

Each LED 22/24 may be housed in its own lamp 20, or all LED's may be housed in a single lamp 20. The two separate LED lamps 20 may be replaced with a single lamp with two LED's such that light output from each LED 22/24 is in alignment with the aperture 76 and input end of the contra angle fiber optic element when its wavelength is selected. There may be one or more optical barriers 38 (FIG. 5) between the lamps 20 to prevent one LED in a first lamp from causing an adjacent LED in a second lamp to fluoresce and emit a 2^{nd} wavelength of light into the contra angle fiber optic element while the first LED is emitting light into the fiber optic element.

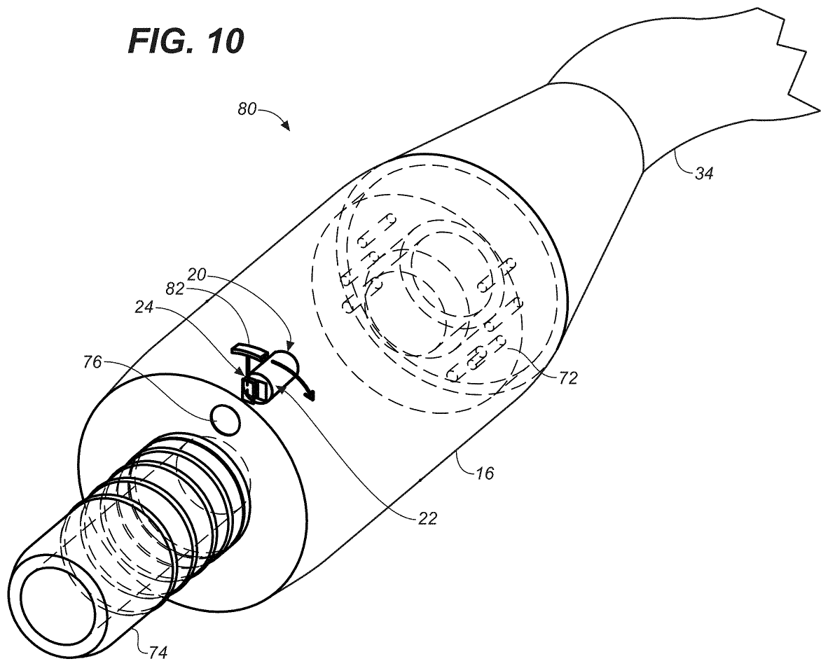

FIG. 10 shows a schematic diagram of an air driven or electric dental handpiece motor 80 having a multi LED lamp 20 with shutter 82. A distal end 74 of handpiece motor 80 detachably connects to an attachable device such as a contra angle comprising a wave guide such as a fiber optic element (both not shown), while the proximal end of the motor body detachably connects to an air/water/electricity supply hose 34.

Two or more single wavelength LED's 22/24 of differing wavelengths are mounted in a single lamp assembly 20 in close proximity to each other and located near aperture 76 on the face of the motor that opposes the contra angle (not shown) when it is connected. Each LED 22/24 is mounted in the lamp 20 in substantial alignment with the aperture 76 and fiber optic element in the contra angle when the contra angle is attached allowing its light output to be efficiently transmitted into and through the fiber optic element of the contra angle. A mechanical, electrical, optical, or other type shutter 82 is located in between the lamp 20 and aperture 76 such that the shutter 82 can be moved or otherwise deployed to prevent the light being emitted by all but one LED to enter the fiber optic element in the contra angle. The LED lamp may or may not be removable by the user.

The motor 80 may also include means (not shown) for user selection of wavelength. In one embodiment, the motor includes means for moving the shutter 82 relative to the position of the LED's 22/24 in the lamp 20. The shutter 82 movement may be linear, elliptical, two dimensional, three dimensional, or any other type of movement that serves to reposition the shutter as needed relative to the LED's. Moving the shutter 82 serves to allow the light emitted by a different LED 22/24 to enter the fiber optic element of the contra angle and thereby changing the wavelength that exits the contra angle near the working instrument (bur, etc.).

Further movement of the shutter 82 may be to deploy an additional wavelength LEDs. The contra angle may be removed by the user in order to access and move the shutter to effect wavelength change. Identifying markings or colors on the motor face may be used by the user to easily move the shutter to the right position for a desired wavelength.

Alternatively, the external surface of the motor 80 may contain a lever, dial, ring or other mechanism (not shown) allowing the user to select a specific LED and wavelength without removing the contra angle. Identifying markings or colors on the motor exterior and/or shared element may be used by the user to easily move the shutter to the right position for a desired wavelength.

In another alternative embodiment, the shutter 82 may be coupled to a powered drive mechanism (motor, etc.) that serves to move the shutter 82 and thereby allow the light emitted by a different LED 22/24 to enter the contra angle fiber optic element and thereby change wavelengths. The powered drive mechanism may be controlled by a switch or other control device (not shown) located on the motor, motor control box, the dental delivery system, or remotely.

Each LED 22/24 mounted to the shared element shall have an electrical connection (e.g. electrical leads, not shown) to the motor electrical supply (e.g. from supply hose 34). The electrical connection may be continuous, and all LED's mounted to the shared element may be powered simultaneously, but only the LED in alignment with the contra angle fiber optic element will transmit light ultimately through the contra angle to the work area.

Alternatively, the electrical connection to each individual LED 22/24 mounted on the shared element may be dynamic such that when a specific LED 22/24 is moved into position in alignment with the contra angle fiber optic element the LED 22/24 changes from unpowered to powered by virtue of its change in position, and the other LED's are unpowered.

In another alternative embodiment, the electrical connection to each individual LED 22/24 mounted on the shared element may be switchable such that when a specific LED 22/24 is moved into position in alignment with the contra angle fiber optic element the LED 22/24 changes from unpowered to powered by virtue of its change in position, and the other LED's are unpowered.

In another alternative embodiment, the shutter 82 could be stationary and the LED lamp 20 moved relative to the shutter 82, achieving the same result. Each LED 22/24 may be housed in its own lamp 20, or all LED's 22/24 may be housed in a single lamp 20.

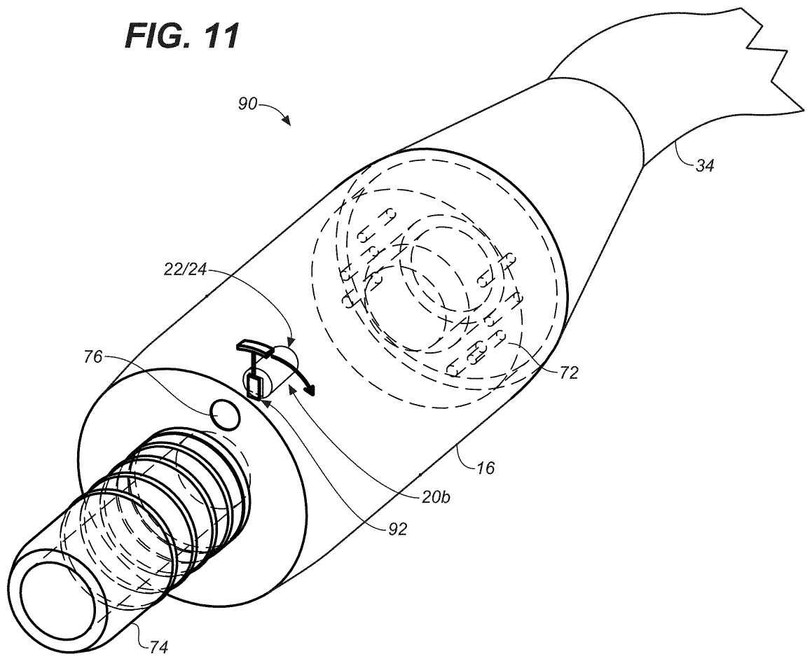

FIG. 11 shows a schematic diagram of an air driven or electric dental handpiece motor 90 having a single lamp 20b with single white or 405 nm LED 22/24 with filter 92. For example, filter may comprise a bandpass filter that only allows light at or near 405 nm when a white lamp is used, or a phosphorous coated lens that emits bright white light with a 405 nm lamp is used. A distal end 74 of handpiece motor 90 detachably connects to an attachable device such as a contra angle comprising a wave guide such as a fiber optic element (both not shown), while the proximal end of the motor body detachably connects to an air/water/electricity supply hose 34.

A single wavelength LED lamp 20b is located near the face of the motor and aperture 76 that opposes the contra angle when it is connected. The LED 22/24 is mounted in the lamp 20b in substantial alignment with the aperture 76 a fiber optic element in the contra angle when the contra angle is attached, allowing its light output to be efficiently transmitted into and through the fiber optic element of the contra angle. A mechanical, electrical, optical, or other type of band pass filter 92 is located between the lamp 20 such that the filter can be moved or otherwise deployed to prevent all the light being emitted by the LED to enter the fiber optic element in the contra angle, except for light of the desired wavelength range (e.g. in the 405 nm, or white light ranges) vicinity.

Wavelength selection means for the filter 92 may be employed similar to that detailed above for shutter 82 of FIG. 10.

In alternative embodiments, other light directing elements (such as one or more mirrors, prisms, lenses, or moveable or bifurcated fiber optic elements, all not shown), may be employed in a configuration using wavelength selection means similar to that detailed above for shutter 82 of FIG. 10 such that the optical element is translated or rotated with respect to LED's 22/24 or lamps 20 to affect varying the light output to the instrument coupled to the handpiece motor. A further alternative embodiment includes uses two small single wavelength LED lamps 20b of different wavelengths (e.g. 405 nm and white) mounted stationary in close proximity to aperture 76. The single wavelength LED lamps 20b may be individually coupled to a switch 12 (e.g. polarity switch shown in FIG. 4a and FIG. 4b) located on the handpiece motor, or control box 16, to allow for selection of one of the different wavelengths operated by the separate lamps 20b. In another embodiment, a single lamp 20b may be employed that has the ability to switch between two or more different wavelengths (e.g. 405 nm and white light).

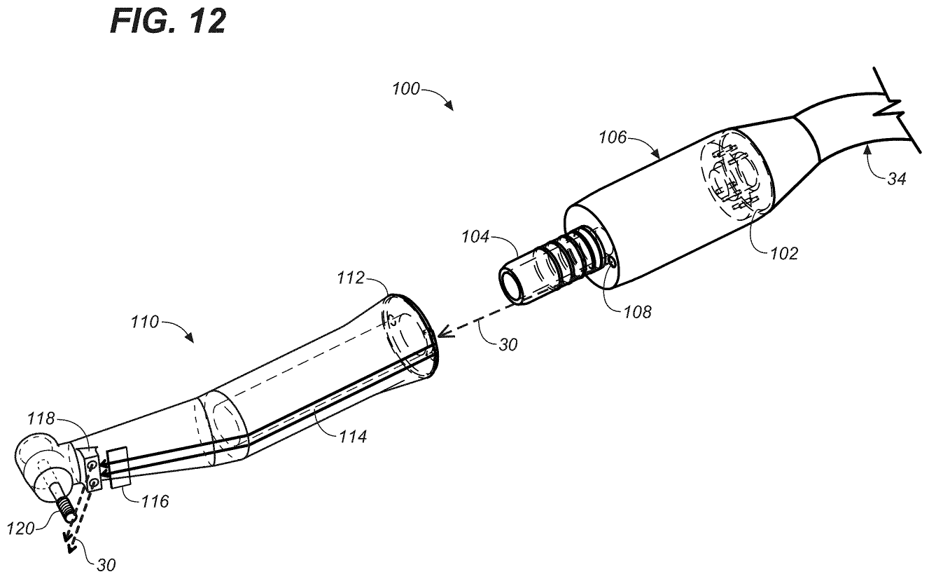

FIG. 12 shows a schematic diagram of a dental assembly 100 comprising a handpiece motor/swivel coupler 106 and air driven or electrical dental handpiece or contra angle 110, where the motor comprises a lamp illumination source 108 for directing white light (or 405 nm light) into a waveguide 114 (e.g. fiber optic element) that traverses the handpiece 110 from proximal coupling end 112 to the distal end at 118. A distal end 104 of motor motor/swivel coupler 106 detachably connects to contra angle/handpiece 110, while the proximal end of the motor body 106 detachably connects to an air/water/electricity supply hose 34.

Along or near the optical path of the fiber optic element 114 is disposed a filter 116, which may be moveable or stationary. Filter 116 may comprise a bandpass filter that only allows light at or near 405 nm when a white lamp is used for illumination source 108, or a phosphorous coated lens that emits bright white light when a 405 nm lamp is used for illumination source 108. Light is emitted at the distal end of the contra angle/handpiece 110 via one or more apertures or lenses 118 that focus an illumination beam 30 at surface surrounding working end cutting instrument 120.

In one embodiment, the filter 116 is movable by employing any one of wavelength selection means similar to those detailed above for shutter 82 of FIG. 10. This enables a single handpiece or contra angle 110 to provide white or 405 nm light output depending on whether the filter 116 is positioned in the path of the illumination light output 30 or retracted from the path.

In another embodiment, a band pass filter lens or coating 116 is applied to the input or output end of the fiber optic element 114 in a first handpiece or contra angle 110 to allow only light in the vicinity of 405 nm wavelength or thereabouts to be omitted. When the dentist wants to deploy only 405 nm light in order to use fluorescence to assist in the removal of caries or tooth colored composite filling as well as other procedures, the dentist detaches the first handpiece or contra angle and attaches a second handpiece/contra angle with the 405 nm bandpass filter 116.

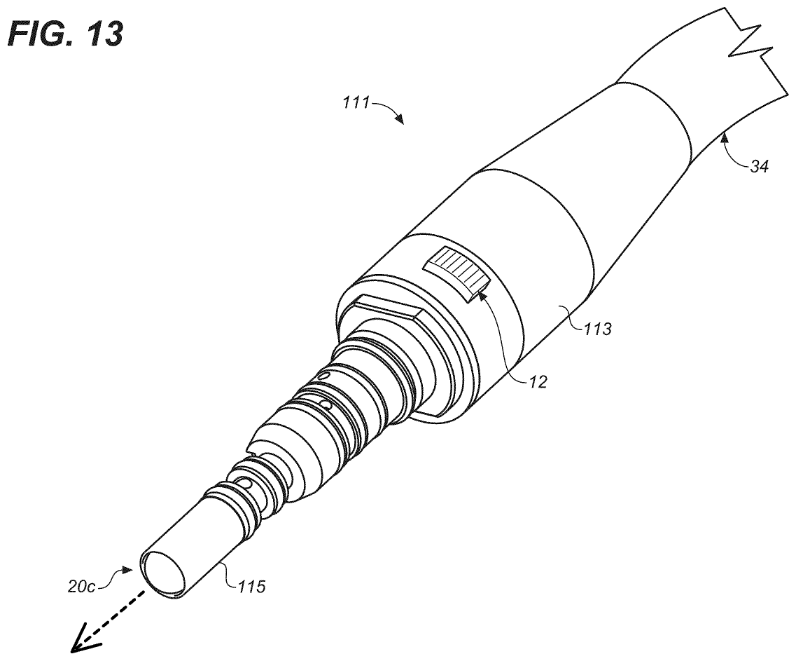

FIG. 13 shows a schematic diagram of a dental handpiece swivel coupler 111 having a single LED lamp 20c with dual wavelength output (405 nm and white). The distal end 115 of the swivel coupler 111 detachably connects to an air driven handpiece with a centered fiber optic element (both not shown) and the proximal end 113 detachably connects to an air/water/electricity supply hose 34. A lamp 20c with a single LED with dual wavelength output (405 nm and white) is mounted near the distal end 115 of the swivel coupler 111 and aligned such that most of the light output of both wavelengths emitted by the single LED, when activated, would enter the fiber optic element in the air driven handpiece when the handpiece is attached. The coupler 111 sends light from each wavelength to enter the fiber optic element in the back end of the air driven handpiece when it is attached to the swivel coupler.

The swivel coupler 113 includes an exterior mounted electrical switch 12 for user selection of wavelength. A circuit arrangement and switching mechanism may be employed in the swivel coupler 111 allowing the user to activate one of the two wavelength LED outputs (white or 405 nm) and to change from a first wavelength to a second wavelength.

Alternatively, a circuit arrangement and switching mechanism in a remote wired or wireless switch (not shown) allows the user to activate one of the two wavelength LED outputs (white or 405 nm) and to change from a first wavelength to a second wavelength. Identifying markings or colors (not shown) in proximity to the switching mechanism 12 may be employed to assist the user in actuating the switch to the right position for the desired white or 405 nm wavelength. There may be one or more optical barriers 38 (FIG. 5) between the different wavelength output areas on the LED to prevent one area from causing an adjacent area to fluoresce and emit a 2^{nd} wavelength of light into the contra angle fiber optic element while the first area is emitting light into the fiber optic element.

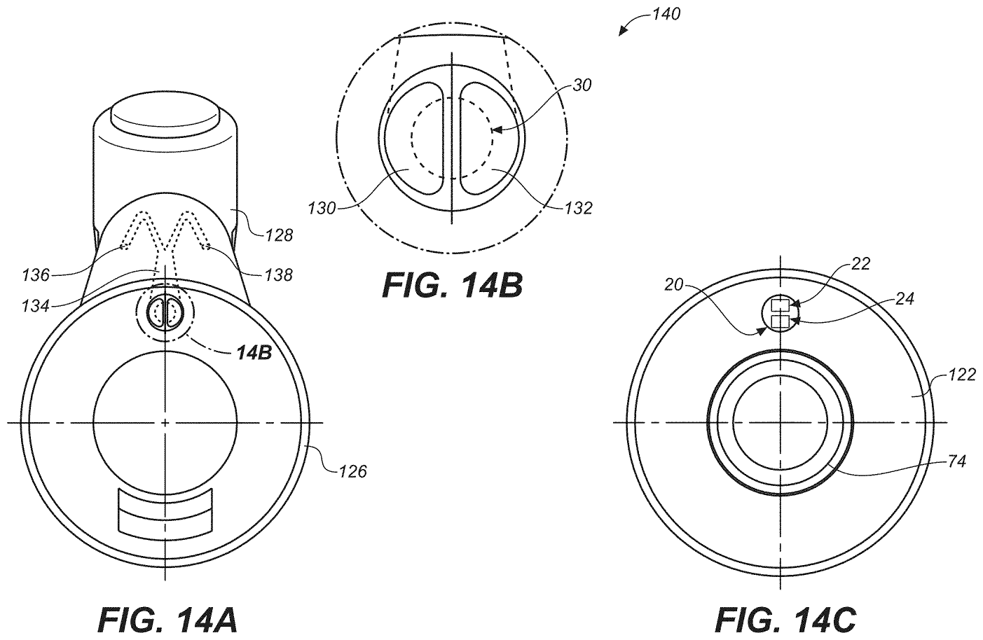

FIG. 14A through FIG. 14C shows a schematic side view of a dental assembly 140 comprising a dental handpiece motor 122 having an orientation of LEDs 22/24 or lamps 20 so as to direct light into a pair of channels 130/132 of contra angle 126 bifurcated fiber optic element 134. Distal end 74 of the motor 122 connects to the proximal end of the mechanically driven contra angle 126 with an off-center fiber optic element 134 (the proximal end of the motor 122 coupling to an air/water/electricity supply hose 34 as shown in FIG. 12). The motor 122 may in turn connect to a motor control box 38 (e.g. FIG. 2). Two single wavelength LED lamps 20 of differing wavelengths (white and 405 nm) or a single lamp 20 with two LEDs 22/24 of differing wavelengths (white and 405 nm) are mounted in close proximity near the face of the motor 122.

The fiber optic element 134 in the contra angle 126 is bifurcated between left 130 and right 132 channels, such that the input (motor) end of the fiber optic element 134 is converged into a single element to receive the light output from the motor, and the output end of the fiber optic element is bifurcated such that two separate output channels 136 and 138 deliver light to the end of the working instrument 128 (e.g. carbide bur, diamond, etc.), thereby reducing shadows. The contra angle fiber optic element 134 at the input end is constructed so that the majority of the area leading to one of the bifurcated output channels 130, 132 is on one side of a dividing line and the majority of the area leading to the second bifurcated output is on the other side of the dividing line. The two LEDs 22/24 or LED lamps are oriented in the motor 122 when positioned to provide their output light 30 (white or 405 nm) to the contra angle fiber optic element 134 such that their light output enters the bifurcated element on both sides of the dividing line, thereby assuring substantially equivalent light output from both channels 136 and 138 of the fiber optic element 134 to the working tip of the working instrument 128. A lens may be mounted in the path of the light output of one or more of the LED's 24/24 so as to cause the light output to enter the bifurcated element 134 on both sides of the dividing line thereby assuring substantially equivalent light output from both channels of the fiber optic element to the working tip of the working instrument.

In another embodiment (not shown) a multi-wavelength LED lamp for use in dental instruments such as an air driven dental handpiece, dental handpiece coupler, dental handpiece air driven motor, dental handpiece electric motor, dental handpiece contra angle, or other dental instrument is equipped with a multi-LED lamp with lens to focus or direct light output to the target. The LED lamp may comprise two or more LED's of two or more wavelengths, or one or more LED's of each wavelength. All LED's may be oriented in the lamp so as to emit their light output in substantially the same direction and towards the same target.

Two or more electrical connections on the lamp apply electrical current to one or more LED's of one wavelength wired in parallel to all the LED's of a second or more wavelengths. The number of electrical connections is equal to the number of wavelengths plus one. LED's are mounted in the lamp at the end of two or more conducting pins housed in a non-conducting material (see FIG. 3). Two or more LED's may be mounted on each conducting pin so as to effectuate a parallel circuitry between LED's of different wavelengths. Alternatively, LED's may be mounted on a circuit board sufficiently small to fit in the handpiece, coupler, motor or contra angle with LED's of different wavelengths wired in a parallel circuitry. Means may be provided for a separable connection of the lamp to the dental instrument by the user. Alternatively, the lamp is not separable by the user from the dental instrument. The lamp may have one or more optical barriers to prevent the output of one LED from causing an adjacent LED(s) to fluoresce and simultaneously provide light output. The lamp may have one or more optical lens serving to direct or focus the output of the LED's towards the target.

In another embodiment (not shown) electric dental handpiece motor and motor control box having two single wavelength led lamps of different wavelengths (405 nm and white) mounted stationary in close proximity, with a single rotatable optical element to direct their output to contra angle optic element. One end of the motor connects to a mechanically driven contra angle with an off-center fiber optic element and the other end connects to an air/water/electricity supply hose that in turn connects to a motor control box. Both are detachable connections. Two single wavelength LED lamps of differing wavelengths (white and 405 nm) are mounted in close proximity near the face of the motor that opposes the contra angle when it is connected and a fiber optic element is positioned in the motor with its input end in alignment with the first of the LED lamps in the motor, and its output end is located at or near the face of the motor in alignment with the contra angle fiber optic element. The fiber optic element has a bend on its input side and a straight section on its output side and can be rotated about the center axis of its straight section.

The motor fiber optic element may be rotated to reposition its input end to be in alignment with the second of the LED lamps in the motor while maintaining alignment of its output end with the contra angle fiber optic element, thereby changing wavelengths of light sent to the contra angle fiber optic element. Lamps may or may not be removable by the user. The LED lamps may be powered simultaneously, or when the motor fiber optic element is rotated to change wavelengths the rotation may be in some way coupled to an electric switch in the motor that has the function of shutting off the lamp no longer in alignment with the motor optic element while activating the lamp in alignment with the motor optic element. The rotation of the motor fiber optic element could be actuated by a mechanism on the face of the motor, or on the external surface of the motor. This could include a lever, dial, band, etc. allowing the user to actuate the mechanism and change wavelengths. Identifying markings or colors in proximity to the actuating mechanism assist the user in rotating the motor fiber optic element to the right position for the desired white or 405 nm wavelength. The two separate LED lamps could be replaced with a single lamp with two LED's with light output from each LED in alignment with the input end of the motor fiber optic element in one of its two rotational positions. There may be one or more optical barriers between the lamps or LED's to prevent one LED in a first lamp from causing an adjacent LED in a second lamp to fluoresce and emit a 2^{nd} wavelength of light into the contra angle fiber optic element while the first LED is emitting light into the fiber optic element.

In a further embodiment (not shown), a swivel coupler for an air driven handpiece, scaler or other dental instrument utilizes two single wavelength LED lamps of different wavelengths (405 nm and white) mounted stationary in a swivel coupler, with single rotatable optic element to direct their output to air driven handpiece optic element. One end of the swivel coupler connects to an air driven dental handpiece or other instrument with a center mounted fiber optic element and the other end connects to an air/water/electricity supply hose. Two or more single wavelength LED lamps of differing wavelengths (white and 405 nm) are mounted in close proximity in the swivel coupler near the end that connects to the supply hose.

A fiber optic element is positioned in the coupler with its input end in alignment with the first of the LED lamps in the coupler, and its output end is located on center line at or near the output end of the coupler in alignment with the air driven handpiece fiber optic element. The fiber optic element has a bend on its input side and a straight section on its output side and can be rotated about the center axis of its straight section. The coupler fiber optic element may be rotated to reposition its input end to be in alignment with the second of the LED lamps in the coupler while maintaining alignment of its output end with the air driven dental handpiece fiber optic element, thereby changing wavelengths of light sent to the handpiece fiber optic element. The lamps may or may not be removable by the user. The LED lamps may be powered simultaneously, or when the coupler fiber optic element is rotated to change wavelengths the rotation may be in some way coupled to an electric switch in the coupler that has the function of shutting off the lamp no longer in alignment with the coupler optic element while activating the lamp in alignment with the coupler optic element. The rotation of the coupler fiber optic element could be actuated by a mechanism near the output end of the swivel coupler, or on the external surface of the coupler near its connection to the supply hose. This could include a lever, dial, band, etc. allowing the user to actuate the mechanism and change wavelengths. Identifying markings or colors in proximity to the actuating mechanism assist the user in rotating the coupler fiber optic element to the right position for the desired white or 405 nm wavelength. The two separate LED lamps could be replaced with a single lamp with two LED's with light output from each LED in alignment with the input end of the coupler fiber optic element in one of its two rotational positions. There may be one or more optical barriers between the lamps or LED's to prevent one LED in a first lamp from causing an adjacent LED in a second lamp to fluoresce and emit a 2^{nd} wavelength of light into the handpiece fiber optic element while the first LED is emitting light into the fiber optic element.

A variation (not shown) to this embodiment may comprise two movable single wavelength LED lamps of different wavelengths (405 nm and white) mounted in a swivel coupler, with a single fixed optical element to direct their output to air driven handpiece optic element. A fiber optic element has a fixed position in the coupler with its input end in alignment with the first of the LED lamps in the coupler, and its output end is located on center line at or near the output end of the coupler in alignment with the air driven handpiece fiber optic element. The fiber optic element may be straight or bent in one or more axes. The LED lamps may be repositioned relative to the input end of the coupler fiber optic element so that the first lamp is no longer in alignment with the coupler fiber optic element and the second lamps is positioned in alignment with the coupler fiber optic element, thereby changing wavelengths of light sent to the handpiece fiber optic element. The LED lamps may be powered simultaneously, or when their positions are changed it may in some way be coupled to an electric switch in the coupler that has the function of shutting off the lamp no longer in alignment with the coupler optic element while activating the lamp in alignment with the coupler optic element. The movement of the LED lamps could be actuated by a mechanism as detailed in various embodiments above.

Another variation (not shown) to the embodiment above may comprise a dental instrument having two single wavelength LED lamps in swivel coupler of different wavelengths (405 nm and white) mounted stationary in close proximity with bifurcated fiber optic element to direct their output to output end of swivel coupler. Two single wavelength LED lamps of differing wavelengths (white and 405 nm) are mounted in close proximity near the end of the coupler that connects to the supply hose and a coupler fiber optic element with the output end converged into a single element and the input end bifurcated into two elements. The bifurcated fiber optic element is positioned such that one of the two ends of the bifurcated input side is aligned to capture most of the light output of each LED lamp, and the single converged output end of the fiber optic element is aligned on the center line of the coupler such that most of the light output of either lamp would enter the fiber optic element in the air driven handpiece when it is attached. When the coupler sends power to one of the two different wavelength (white or 405 nm) LED lamps, the light output from that lamp is transmitted through the bifurcated fiber optic element to then enter the fiber optic element in the back end of the air driven handpiece.

The coupler may include an exterior mounted electrical switch for user selection of wavelength or other wavelength selection means as described above. A circuit arrangement and switching mechanism in the coupler effectuates a reversing of polarity, thereby activating only one of the LED lamps and changing from a first wavelength to a second wavelength. Alternatively, a circuit arrangement and switching mechanism in the coupler that effectuates a break in the circuit for a first wavelength LED lamp and closes the circuit for a second wavelength's LED lamp. Alternatively, the switching mechanism could be a wired or wireless remotely mounted switch. Identifying markings or colors in proximity to the switch assist the user in actuating the switch to the right position for the desired white or 405 nm wavelength. The two separate LED lamps could be replaced with a single lamp with two LED's with light output from each LED in alignment with one of the bifurcated ends of the fiber optic element. There may be one or more optical barriers between the LEDs to prevent one LED in a first lamp from causing an adjacent LED in a second lamp to fluoresce and emit a 2^{nd} wavelength of light into the contra angle fiber optic element while the first LED is emitting light into the fiber optic element. Alternatively, in lieu of an electrical switch, a mechanical or optical shutter could be connected to an actuation mechanism and the shutter could be moved to block the output of one LED lamp and allow the other to radiate, thereby changing wavelengths. Alternatively, in lieu of an electrical switch or a shutter, a mirror could be connected to an actuation mechanism and the mirror could be moved to block the output of one LED lamp and allow the other to radiate, thereby changing wavelengths.

In addition to the above embodiments, variations (not shown) may include an air driven dental handpiece swivel coupler having a dual LED lamp (or two lamps) with a shutter, filter, coated lens or mirror (as detailed in various embodiments above) to select wavelength, with the LED lamp containing two or more LED's of differing wavelengths mounted at or near the handpiece end of the swivel coupler. The coupler sends light from each wavelength LED into fiber optic element in the center of the handpiece back end when the handpiece is attached to the coupler. The coupler includes a movable shutter for user selection of wavelength. The shutter is mounted on the coupler between the LED lamp and the handpiece fiber optic element. When the shutter is moved the output of one LED is blocked and the second LED output is allowed to enter the handpiece fiber optic element, thereby changing wavelengths. A mechanism, as detailed in the various wavelength selection means detailed above, allows the user to move the shutter so as to change wavelengths.

A further embodiment (not shown) includes an air driven dental handpiece swivel coupler having a swivel coupler with dual LED lamp (or two lamps), and fixed fiber optic element with shutter or mirror to select wavelength. An LED lamp containing two or more LED's of differing wavelengths is mounted near the supply hose end of the swivel coupler. The coupler has a fiber optic element with its input side aligned with the output of the LED's and its output side aligned on the coupler center line and the handpiece fiber optic element. The LED's and lamp are positioned so that the output of both LED's is aimed to enter the coupler fiber optic element. The coupler sends light from each wavelength LED into fiber optic element in the center of the handpiece back end when the handpiece is attached to the coupler.

The coupler includes a movable shutter for user selection of wavelength. The shutter is mounted in the coupler between the LED lamp and the coupler fiber optic element. When the shutter is moved the output of one LED is blocked and the second LED output is allowed to enter the coupler fiber optic element, thereby changing wavelengths. A mechanism allows the user to move the shutter so as to change wavelengths. The mechanism may be a ring, knob, lever, etc. mounted on the exterior surface of the swivel coupler. Identifying markings or colors on the coupler exterior assist the user in moving the mechanism to the right position for a desired wavelength. Electrical power to the swivel coupler is supplied by the supply hose or an air driven electrical generator. Alternatively, instead of a shutter, the swivel coupler could include a movable mirror to effect change of wavelength. Alternatively, instead of a single LED lamp with two LED's, the swivel coupler could include two single wavelength LED lamps of different wavelengths mounted in close proximity.

Another embodiment (not shown) comprises an air driven dental handpiece swivel coupler comprising a single white LED lamp at end of swivel coupler with bandpass filter to select wavelength. One end connects to an air driven dental handpiece or other instrument, the other end to an air/water/electricity supply hose. Both are detachable connections. A white LED lamp is mounted at or near the handpiece end of the swivel coupler. The lamp may or may not be removable by the user. The coupler sends light from the white LED into fiber optic element in the center of the handpiece back end when the handpiece is attached to the coupler. The coupler includes a movable bandpass filter for user selection of wavelength. The filter is mounted on the coupler between the LED lamp and the handpiece fiber optic element. When the filter is moved into the path of the white LED output, all light except light with a wavelength in the vicinity of 405 nm is blocked and only 405 nm light is allowed to enter the handpiece fiber optic element, thereby changing wavelengths. A mechanism allows the user to move the filter so as to change wavelengths. The mechanism may be a ring, knob, lever, etc. mounted on the exterior surface of the swivel coupler. Identifying markings or colors on the coupler exterior assist the user in moving the mechanism to the right position for a desired wavelength. Electrical power to the swivel coupler is supplied by the supply hose or an air driven electrical generator.

A further embodiment (not shown) comprises air driven dental handpiece swivel coupler having a swivel coupler with white LED lamp, and fixed fiber optic element with bandpass filter to select wavelength. One end connects to an air driven dental handpiece or other instrument, the other end to an air/water/electricity supply hose. Both are detachable connections. A single wavelength white LED lamp is mounted near the supply hose end of the swivel coupler. The lamp may or may not be removable by the user. The coupler has a fiber optic element with its input side aligned with the output of the LED lamp and its output side aligned on the coupler center line and the handpiece fiber optic element. The LED lamp is positioned so that the output of the LED is aimed to enter the coupler fiber optic element. The coupler sends light from the LED into fiber optic element in the center of the handpiece back end when the handpiece is attached to the coupler.

The coupler includes a movable bandpass filter for user selection of wavelength. The filter is mounted in the coupler between the white LED lamp and the coupler fiber optic element. When the filter is moved into the path of the white LED output, all light except light with a wavelength in the vicinity of 405 nm is blocked and only 405 nm light is allowed to enter the coupler fiber optic element, thereby changing wavelengths. A mechanism allows the user to move the filter so as to change wavelengths. The mechanism may be a ring, knob, lever, etc. mounted on the exterior surface of the swivel coupler. Identifying markings or colors on the coupler exterior assist the user in moving the mechanism to the right position for a desired wavelength. Electrical power to the swivel coupler is supplied by the supply hose or an air driven electrical generator.

In a further embodiment (not shown), an air driven dental handpiece swivel coupler comprises a single 405 nm LED lamp at end of swivel coupler with phosphorous coated lens to select wavelength. The coupler sends light from the 405 nm LED into fiber optic element in the center of the handpiece back end when the handpiece is attached to the coupler.

The coupler includes a movable phosphorous coated lens for user selection of wavelength. The lens is mounted on the coupler between the LED lamp and the handpiece fiber optic element. When the lens is moved into the path of the 405 nm LED output, the 405 nm light causes the phosphorous to fluoresce white light which then enters the handpiece fiber optic element, thereby changing wavelengths

A further embodiment comprises a swivel coupler with 405 nm LED lamp, and fixed fiber optic element with phosphorous lens to select wavelength. The coupler has a fiber optic element with its input side aligned with the output of the LED lamp and its output side aligned on the coupler center line and the handpiece fiber optic element. The LED lamp is positioned so that the output of the LED is aimed to enter the coupler fiber optic element. The coupler sends light from the LED into fiber optic element in the center of the handpiece back end when the handpiece is attached to the coupler.

The coupler includes a movable phosphorous coated lens for user selection of wavelength. The lens is mounted in the coupler between the 405 nm LED lamp and the coupler fiber optic element. When the lens is moved into the path of the 405 nm LED output, the 405 nm light causes the phosphorous to fluoresce white light which then enters the handpiece fiber optic element, thereby changing wavelengths. A mechanism allows the user to move the filter so as to change wavelengths.

An additional embodiment comprises a swivel coupler with two single wavelength LED lamps and electrical switch to change wavelengths. The two LED lamps are mounted in close proximity and aligned such that both their light outputs are aimed at the fiber optic element in the center of the handpiece back end. The coupler sends light from each wavelength LED lamp into fiber optic element in the center of the handpiece back end when the handpiece is attached to the coupler.

The coupler includes an electrical switch for user selection of wavelength. A circuit arrangement and switching mechanism in the coupler that effectuates a reversing of polarity, thereby causing only one LED lamp to emit light and changing from a first wavelength to a second wavelength LED lamp. Alternatively, a circuit arrangement and switching mechanism in the coupler that effectuates a break in the circuit for a first wavelength LED lamp and closes the circuit for a second wavelength's LED lamp. A ring, band, knob, lever or other mechanism on the external surface of the swivel coupler allows the user to actuate the switch and change wavelengths. Identifying markings or colors on the coupler exterior assist the user in moving the mechanism to the right position for a desired wavelength. Electrical power to the swivel coupler is supplied by the supply hose or an air driven generator internal or external to the swivel coupler, the air provided by the supply hose to the coupler or returning from the handpiece.

An additional embodiment comprises a swivel coupler with two single wavelength LED lamps, fiber optic element, and electrical switch to change wavelengths. Two single wavelength LED lamps each containing LED's of differing wavelengths is mounted at or near the supply hose end of the swivel coupler. A fiber optic element in the swivel coupler transmits the light from both LED lamps to the fiber optic element in the center of the handpiece back end. The two LED lamps are mounted in close proximity and aligned such that both their light outputs are aimed at the input end of the fiber optic element in the swivel coupler. The coupler sends light from each wavelength LED lamp into fiber optic element in the center of the handpiece back end when the handpiece is attached to the coupler.

From the description herein, it will be appreciated that the present disclosure encompasses multiple embodiments which include, but are not limited to, the following:

1. An air driven dental handpiece swivel coupler, comprising:

a. One end connects to an air driven dental handpiece, the other end to an air/water/electricity supply hose. Both are detachable connections.

b. An LED lamp containing two LEDs of differing wavelengths—white and 405 nm—is mounted at or near the handpiece end of the swivel coupler.

b1. Lamp may or may not be removable by the user.

c. Coupler emits light from each wavelength LED into fiber optic element in the center of the handpiece back end when the handpiece is attached to the coupler.

d. Coupler includes electrical switch for user selection of wavelength:

d1. A circuit arrangement and switching mechanism in the coupler that effectuates a reversing of polarity, thereby changing from a first wavelength to a second wavelength; or

d2. A circuit arrangement and switching mechanism in the coupler that effectuates a break in the circuit for a first wavelength LED(s) and closes the circuit for a second wavelength's LED(s).

d3. A switch activation member on the external surface of the coupler that the user moves from a first position A to a second position B to change wavelength, such switch being actuated by the user without risk of loosening the supply hose nut on the coupler:

d3i. button, rocker, slider or other small switching mechanism.

d4. Identifying markings, letters or colors on the coupler exterior assist the user in moving the mechanism to the desired position to change from a first wavelength to the second wavelength.

d4i. Polarity is not known until installation, so the markings do not indicate wavelength (blue or white), only A or B wavelength.

e. Electrical power to the swivel coupler is supplied by:

e1. The supply hose; or

e2. an air driven generator internal or external to the swivel coupler, the air provided by the supply hose to the coupler or returning from the handpiece.

2. An electric dental handpiece motor and motor control box, comprising:

a. One end of the motor connects to a mechanically driven contra angle with an off-center fiber optic element and the other end connects to an air/water/electricity supply hose that in turn connects to a motor control box. Both are detachable connections.

b. An LED lamp containing two LEDs of differing wavelengths (white and 405 nm) is mounted in/on the face of the motor that opposes the contra angle when it is connected and:

b1. Is mounted off center and in alignment with the fiber optic element in the contra angle when the contra angle is attached.

b1i. Lamp may or may not be removable by the user.

c. Motor sends light from each wavelength energized LED to enter the fiber optic element in the back end of the contra angle when the contra angle is attached to the motor.

d. Motor control box includes an electrical switch for user selection of wavelength:

d1. A circuit arrangement and switching mechanism in the motor control box effectuates a reversing of polarity, thereby changing from a first wavelength to a second wavelength; or

d2. A circuit arrangement and switching mechanism in the motor that effectuates a break in the circuit for a first wavelength LED(s) and closes the circuit for a second wavelength's LED(s).

d3. Identifying markings or colors on the motor control box exterior in in the motor box interface in proximity to the switch assist the user in actuating the switch to the right position for the desired white or 405 nm wavelength.

3. Air driven dental handpiece or mechanically driven contra angle, comprising:

a. One end connects to air/water/supply hose or motor, with or without electricity, with a detachable connection.

b. Dual wavelength (white and 405 nm) LED lamp mounted in close proximity of working instrument (bur, etc.) and means to transmit electrical power from the source of such power to the LED lamp:

b1. When handpiece/contra angle is activated one wavelength light is emitted near/around area of working instrument contact.

b1i. Lamp may or may not be removable by the user.

c. Handpiece/contra angle includes exterior mounted electrical switch for user selection of wavelength:

c1. A circuit arrangement and switching mechanism in the handpiece/contra angle that effectuates a reversing of polarity, thereby changing from a first wavelength to a second wavelength; or

c2. A circuit arrangement and switching mechanism in the handpiece/contra angle that effectuates a break in the circuit for a first wavelength LED(s) and closes the circuit for a second wavelength's LED (s).

c3. A switch on the external surface of the handpiece/contra angle that the user actuates from a first position to a second position:

c4. Identifying markings, letters, or colors on the handpiece/contra angle exterior assist the user in moving the mechanism to the right position for a desired wavelength.

d. Power to LEDs supplied by:

d1. Electrical generator (fluid driven or mechanically driven) in handpiece/contra angle; OR

d2. Power from air/water/electrical supply hose

d2i. Static separable connection, not swivel; OR

d2ii. Rotating dynamic connection. Swivel and separable.

4. A dual-wavelength (white and 405 nm) LED lamp for use in dental instruments such as an air driven handpiece, handpiece coupler, handpiece air driven motor, handpiece electric motor, or handpiece contra angle, powered or manual scaler, or other dental instrument, comprising:

a. LEDs of two different wavelengths, 405 nm and white.

a1. One or more LEDs of each wavelength.

b. All LEDs oriented in the lamp so as to emit their light output in substantially the same direction and towards the same target.

c. LEDs small enough, arranged in sufficient density, with sufficient output intensity, and arranged in a manner that allows them to emit enough light into the corresponding light guide of a diameter not to exceed 0.125 inches so as to accomplish the intended purpose of that wavelength (excitation, illumination, etc.)

d. Two electrical connections on the lamp for applying electrical current to one or more LEDs of one wavelength wired in parallel to all the LEDs of the second wavelength.

e. LEDs mounted in the lamp:

e1. At the end of two or more conducting pins housed in a non-conducting material

e1i. Two or more LEDs mounted on each conducting pin so as to effectuate a parallel circuitry between LEDs of different wavelengths; or

e1ii. On a circuit board sufficiently small to fit into the dental instrument with LEDs of different wavelengths wired in a parallel circuitry.

f. The lamp may or may not be removable by the user.

g. Autoclavable lamp components.

5. A dental instrument utilizing two or more wavelength light outputs for the purposes of excitation, illumination, etc. of the work area with external markings on said instrument allowing the user to see before beginning work which wavelength the instrument is set on (e.g., white, 405 nm, etc.) and also enabling the user before or during a procedure to actuate a mechanism (ring, lever, etc.) to a specific position or setting to change the selected wavelength the instrument will emit.

a. The markings, dots or figures are the color of the selected wavelength (i.e. white for illumination and blue for 405 nm).

b. OR the markings are symbolic representations of the application (excitation, illumination, etc.) specific to the wavelength; OR

c. The markings are words or abbreviations of words such as

WHITE or WHT for illumination and BLUE or BL for 405 nm).

6. A scaler or other powered dental instrument for removing plaque, calculus or tartar from teeth, comprising:

a. Electrical power supplied to the instrument by supply hose at the non-working end of the instrument.

b. A working tip on the working end of the instrument that comes in contact with the tooth during the removal of plaque, etc.

c. One or more white and 405 nm LEDs are arranged on the instrument so as to illuminate the area of the tooth surrounding the working tip sufficiently as to allow the user:

c1. To aid in the visualization of remaining plaque, etc. and/or

c2. to direct the working tip to areas of the tooth with remaining plaque, etc.

d. Means for transmitting the electrical power from the non-working end of the instrument to the LEDs located in the vicinity of the working tip.

7. A scaler or other powered dental instrument for removing plaque, calculus or tartar from teeth, comprising:

a. Means on the non-working end of the instrument for connection to a supply hose providing air and water.

b. A fluid activated generator located in the non-working vicinity of the instrument.

c. A working tip on the working end of the instrument that comes in contact with the tooth during the removal of plaque, etc.

d. One or more white and 405 nm LEDs are arranged on the instrument so as to illuminate the area of the tooth surrounding the working tip sufficiently as to allow the user:

d1. to aid in the visualization of remaining plaque, etc. and/or

d2. to direct the working tip to areas of the tooth with remaining plaque, etc.

e. Means for transmitting the electrical power from the generator to the LEDs located in the vicinity of the working tip.

8. A scaler or other powered dental instrument for removing plaque, calculus or tartar from teeth, comprising:

a. Means on the non-working end of the instrument for connection to a supply hose providing air, water and a source of white and 405 nm light.

b. A working tip on the working end of the instrument that comes in contact with the tooth during the removal of plaque, etc.

c. A light guide in the instrument for transmitting white and 405 nm light from the non-working end of the instrument to illuminate the area of the tooth surrounding the working tip sufficiently as to allow the user:

c1. to see remaining plaque, etc. and/or

c2. to direct the working tip to areas of the tooth with remaining plaque, etc.

9. A scaler or other powered dental instrument for removing plaque, calculus or tartar from teeth, comprising:

a. Means on the non-working end of the instrument for connection to a supply hose providing air, water, and electrical power.

b. One or more white and 405 nm LEDs located in the instrument in the vicinity of the non-working end of the instrument.

c. A working tip on the working end of the instrument that comes in contact with the tooth during the removal of plaque, etc.

d. A light guide in the instrument for transmitting white and 405 nm light from the LEDs housed in the non-working end of the instrument to illuminate the area of the tooth surrounding the working tip sufficiently as to allow the user:

d1. to see remaining plaque, etc. and/or

d2. to direct the working tip to areas of the tooth with remaining plaque, etc.

10. A scaler or other powered dental instrument for removing plaque, calculus or tartar from teeth, comprising:

a. Means on the non-working end of the instrument for connection to a supply hose providing air and water.

b. One or more white and 405 nm LEDs housed in the vicinity of the non-working end of the instrument.

c. A fluid activated generator located in the instrument in the vicinity of the non-working end of the instrument to supply electrical power to the 405 nm LEDs.

d. A working tip on the working end of the instrument that comes in contact with the tooth during the removal of plaque, etc.

e. A light guide in the instrument for transmitting white and 405 nm light from the LEDs housed in the non-working end of the instrument to illuminate the area of the tooth surrounding the working tip sufficiently as to allow the user:

e1. to see remaining plaque, etc. and/or

e2. to direct the working tip to areas of the tooth with remaining plaque,

11. A manually powered dental instrument for removing plaque, calculus or tartar from teeth, comprising:

a. Electrical power supplied to the instrument by battery:

a1. Replaceable but not rechargeable; or

a2. Rechargeable.

b. A working tip on the working end of the instrument that comes in contact with the tooth during the removal of plaque, etc.

c. One or more white and 405 nm LEDs are arranged on the instrument so as to illuminate the area of the tooth surrounding the working tip sufficiently as to allow the user:

c1. to see remaining plaque, etc. and/or

c2. to direct the working tip to areas of the tooth with remaining plaque, etc.

d. Means for transmitting the electrical power from the battery to the LEDs located in the vicinity of the working tip.

12. A method of removing plaque, calculus or tartar from the tooth surface utilizing a powered or manual dental instrument enabled to emit 405 nm wavelength light whereby 405 nm light emitted from a dental instrument is used to detect plaque, calculus or tartar from the tooth simultaneous with its removal.

13. A method of restoring a tooth utilizing a dental handpiece capable of emitting dual wavelength light whereby:

a. 405 nm light emitted from the dental handpiece is first used to detect and remove decay

b. Restorative material(s) are applied to the tooth.

c. 405 nm light is used to enhance visual differentiation between excess restorative material(s) and tooth while the excess restorative material(s) are removed.

d. The wavelength emitted from the dental handpiece is changed from 405 nm to white light

e. The white light is used to assist in the occlusal adjustment and final surface finishing of the restoration.

14. An air driven dental handpiece swivel coupler apparatus, the apparatus comprising: a swivel coupler; said coupler having a first end configured to connect to an air driven dental handpiece; said coupler having a second end configured to connect to an air/water/electricity supply hose; wherein one or both of said ends comprise detachable connections; an LED lamp containing two LEDs of differing wavelengths mounted at or near the first end of the swivel coupler; wherein the coupler is configured to emit light from each wavelength LED into a fiber optic element in the handpiece when the handpiece is attached to the coupler; and means for selection of wavelength.

15. An electric dental handpiece motor and motor control box apparatus, the apparatus comprising: an electric dental handpiece motor and motor control box; said motor having a first end configured for connection to a mechanically driven contra angle with an off center fiber optic element; said motor having a second end configured for connection to an air/water/electricity supply hose that in turn connects to the motor control box; wherein one or both connections are detachable connections; an LED lamp containing two LEDs of differing wavelengths mounted in/on a face of the motor that opposes the contra angle when it is connected; wherein the lamp is mounted off center and in alignment with the fiber optic element in the contra angle when the contra angle is attached; and means for selection of wavelength.

16. An air driven dental handpiece or mechanically driven contra angle apparatus, the apparatus comprising: an air driven dental handpiece or mechanically driven contra angle having an end configured for connection to an air/water/supply hose or motor, with or without electricity, with a detachable connection; a dual wavelength LED lamp mounted in the air driven dental handpiece or mechanically driven contra angle; and means for selection of wavelength.

17. A dual-wavelength (white and 405 nm) LED lamp apparatus for use in dental instruments, such as an air driven handpiece, handpiece coupler, handpiece air driven motor, handpiece electric motor, or handpiece contra angle, powered or manual scaler, or other dental instrument, the apparatus comprising: a dual-wavelength LED lamp; said lamp having at least one LED for each wavelength; said LEDs oriented in the lamp so as to emit their light output in substantially the same direction and towards the same target; wherein said LEDs are configured in sufficient density and have sufficient output intensity to emit enough light into a light guide of a diameter not to exceed about 0.125 inches.

18. In a dental instrument, such as an air driven handpiece, handpiece coupler, handpiece air driven motor, handpiece electric motor, or handpiece contra angle, powered or manual scaler, or other dental instrument, the improvement comprising: a dual-wavelength LED lamp for providing excitation, illumination, etc. to a work area; and external markings on the instrument that allow the user to see before beginning work which wavelength the lamp is set to emit before activating the lamp.

19. An apparatus for removing plaque, calculus or tartar from teeth, the apparatus comprising: a scaler or other powered dental instrument for removing plaque, calculus or tartar from teeth; a dual wavelength LED lamp mounted on the scaler or other air powered dental instrument; and means for selection of wavelength.

20. A method of removing plaque, calculus or tartar from the tooth surface utilizing a powered or manual dental instrument enabled to emit 405 nm wavelength light whereby a 405 nm light emitted from a dental instrument is used to detect plaque, calculus or tartar from the tooth simultaneous with its removal.

21. A method of restoring a tooth utilizing a dental handpiece capable of emitting dual wavelength light whereby 405 nm light emitted from the dental handpiece is first used to detect and remove decay, restorative material(s) are then applied to the tooth, a 405 nm light is used to enhance visual differentiation between excess restorative material(s) and tooth while the excess restorative material(s) are removed, the wavelength emitted from the dental handpiece is changed from 405 nm to white light, and the white light is used to assist in the occlusal adjustment and final surface finishing of the restoration.

22. The apparatus or method of any of the preceding or subsequent embodiments, further comprising: a physical non-light transmitting optical barrier located between the two LEDs; wherein the optical barrier is configured to shield the non-powered LEDs from radiation emitted by the adjacent LEDs; wherein fluorescence of non-powered LED is minimized or inhibited as a result of said barrier, thus minimizing or inhibiting emission of any non-selected wavelength along with the selected wavelength.

23. The apparatus or method of any of the preceding or subsequent embodiments, wherein the barrier comprises a non-conducting material such as epoxy.

24. The apparatus or method of any of the preceding or subsequent embodiments, wherein the barrier is flat, curved or fully or partially cylindrical.

25. The apparatus or method of any of the preceding or subsequent embodiments, wherein the optical barrier comprises a non-light transmitting coating applied to all or a portion of the external surface(s) of one or both wavelength LEDs.

26. An air driven or electric dental handpiece motor having multiple lamps with a single LED at different radial distances from motor centerline.

27. An air driven or electric dental handpiece motor having a single lamp or multi lamp multi LED rotating about the lamp CTR with axis of rotation parallel or perpendicular to center line of motor.

28. An air driven or electric dental handpiece motor having a single lamp multi LED with a shutter.

29. An air driven or electric dental handpiece motor having a single lamp with single white LED with a bandpass filter.

30. An air driven or electric dental handpiece motor having a single lamp with single 405 nm LED with a phosphorous coated lens to create white light.

31. An air driven dental handpiece or electric handpiece contra angle with fiber optic lighting and bandpass filtering having a bandpass filter on handpiece or contra-angle, front, rear, retractable.

32. An air driven or electric dental handpiece motor having a single lamp dual LED with mirror.

33. An electric dental handpiece motor and motor control box having two small single wavelength LED lamps of different wavelengths (405 nm and white) mounted stationary in close proximity.

34. An electric dental handpiece motor and motor control box having a single LED with dual wavelength output (405 nm and white).

35. A dental handpiece swivel coupler having a single LED with dual wavelength output (405 nm and white).

36. An electric dental handpiece motor and motor control box with two single wavelength led lamps of different wavelengths (405 nm and white) mounted stationary in close proximity, with bifurcated optical element to direct their output to contra angle optic element.

37. An electric dental handpiece motor and motor control box or swivel coupler having an orientation of LEDs or lamps so as to direct light into both channels of contra angle bifurcated fiber optic element.

38. A multi-wavelength LED lamp for use in dental instruments such as an air driven dental handpiece, dental handpiece coupler, dental handpiece air driven motor, dental handpiece electric motor, dental handpiece contra angle, or other dental instrument with a multi-LED lamp with lens to focus or direct light output to the target.

39. A schematic diagram of an electric dental handpiece motor and motor control box having two single wavelength led lamps of different wavelengths (405 nm and white) mounted stationary in close proximity, with a single rotatable optical element to direct their output to contra angle optic element.

40. A swivel coupler for an air driven handpiece, scaler or other dental instrument having two single wavelength LED lamps of different wavelengths (405 nm and white) mounted stationary in a swivel coupler, with single rotatable optic element to direct their output to air driven handpiece optic element.

41. A swivel coupler for an air driven handpiece, scaler or other dental instrument having two movable single wavelength LED lamps of different wavelengths (405 nm and white) mounted in a swivel coupler, with single fixed optical element to direct their output to air driven handpiece optic element.

42. A swivel coupler for an air driven handpiece, scaler or other dental instrument having two single wavelength LED lamps in swivel coupler of different wavelengths (405 nm and white) mounted stationary in close proximity, with bifurcated fiber optic element to direct their output to output end of swivel coupler.

43. An air driven dental handpiece swivel coupler having a dual LED lamp (or two lamps) with shutter or mirror to select wavelength.

44. An air driven dental handpiece swivel coupler having a swivel coupler with dual LED lamp (or two lamps), and fixed fiber optic element with shutter or mirror to select wavelength.

45. An air driven dental handpiece swivel coupler comprising a single white led lamp at end of swivel coupler with bandpass filter to select wavelength.

46. An air driven dental handpiece swivel coupler having a swivel coupler with white LED lamp, and fixed fiber optic element with bandpass filter to select wavelength.

47. An air driven dental handpiece swivel coupler with a single 405 nm LED lamp at end of swivel coupler with phosphorous coated lens to select wavelength.

48. An air driven dental handpiece swivel coupler having a swivel coupler with 405 nm LED lamp, and fixed fiber optic element with phosphorous lens to select wavelength.

49. An air driven dental handpiece swivel couple having a swivel coupler with two single wavelength LED lamps and electrical switch to change wavelengths.

50. An air driven dental handpiece swivel coupler having a swivel coupler with two single wavelength LED lamps, fiber optic element, and electrical switch to change wavelengths.

51. A dental apparatus, comprising: an elongate, handheld housing comprising a proximal end for releasably coupling to a supply hose, the supply hose delivering one or more of power, air or water to the housing; a working instrument disposed on or connected to a distal end of the handheld housing; the working instrument configured for operating on a tooth surface of a patient; one or more illumination sources coupled to the housing; the one or more illumination sources providing an illumination beam at or near the working instrument and tooth surface; and a switching mechanism coupled to the illumination source; the switching mechanism configured to change a wavelength of the illumination beam from a first wavelength to a second wavelength.

52. The dental apparatus or method of any preceding or subsequent embodiment, wherein the first and second wavelengths comprise white light and 405 nm light.

53. The dental apparatus or method of any preceding or subsequent embodiment: wherein the one or more illumination sources comprises an illumination lamp having a first LED illuminating at the first wavelength and a second LED illuminating light at the second wavelength; and wherein said LEDs are oriented in the lamp so as to emit light output in to generate the illumination beam at substantially a same surface area on said tooth surface.

54. The dental apparatus or method of any preceding or subsequent embodiment, wherein the first and second LEDs are configured to emit at a sufficient output density and intensity to emit enough light into a light guide of a diameter not to exceed about 0.125 inches.

55. The dental apparatus or method of any preceding or subsequent embodiment, wherein the switching mechanism comprises an electrical switch that switches a polarity of power delivered to the first LED and second LED, thereby switching the wavelength of the illumination beam from the first LED at the first wavelength to the second LED at the second wavelength.

56. The dental apparatus or method of any preceding or subsequent embodiment: wherein the lamp comprises an optical barrier disposed between the first LED and the second LED; the optical barrier configured to shield a non-powered LED from radiation emitted by an adjacent LED; and wherein fluorescence of the non-powered LED is minimized or inhibited as a result of said barrier, thus minimizing or inhibiting emission of a non-selected wavelength along with a selected wavelength.

57. The dental apparatus or method of any preceding or subsequent embodiment: wherein the one or more illumination sources comprises a first illumination source operating at a first wavelength and a second illumination source spaced apart from the first operating at a second wavelength; and wherein the switching mechanism comprises a mechanical switch that shifts output of light emitted from the first and second illumination sources with respect to the handheld housing such that only one of the first and second illumination sources contributes to illumination beam.

58. The dental apparatus or method of any preceding or subsequent embodiment, wherein the mechanical switch shifts orientation of the first and second illumination sources such that only one of the first and second illumination sources contributes to the illumination beam.

59. The dental apparatus or method of any preceding or subsequent embodiment, wherein the mechanical switch shifts orientation of light output of the first and second illumination sources such that only one of the first and second illumination sources contributes to the illumination beam.

60. The dental apparatus or method of any preceding or subsequent embodiment, wherein the mechanical switch comprises a manually operated lever that switches from a first position to a second position, each corresponding to illumination at respective first and second wavelengths.

61. The dental apparatus or method of any preceding or subsequent embodiment: the housing further comprising an optical element comprising one or more of a lens, mirror, shutter or waveguide; wherein the switching mechanism shifts orientation of the optical element such that only one of the first and second illumination sources contributes to illumination beam

62. The dental apparatus or method of any preceding or subsequent embodiment: wherein the one or more illumination sources comprises a single illumination source operating at a first wavelength; the housing further comprising a filter disposed within a path of light output from the single illumination source; wherein the switching mechanism is configured to shift orientation of the filter with respect to the light output from the single illumination source such that the filter alters the light output from the first wavelength to the second wavelength.

63. The dental apparatus or method of any preceding or subsequent embodiment, wherein one or more illumination sources are powered from the supply hose.

64. The dental apparatus or method of any preceding or subsequent embodiment, wherein one or more illumination sources are powered from a battery disposed within the housing.

65. The dental apparatus or method of any preceding or subsequent embodiment, the housing further comprising a generator configured to convert motive force from air or water supplied from the supply hose to electrical power used to power the one or more illumination sources.

66. The dental apparatus or method of any preceding or subsequent embodiment, further comprising: a dental handpiece having a proximal handpiece end and distal handpiece end housing the working instrument; wherein the housing comprises a coupler having a proximal coupler end for releasably coupling to the supply hose and a distal coupler end for releasably connecting to the proximal handpiece end of the dental handpiece; wherein the one or more illumination sources are configured to output light at one or more of the first and second wavelengths at said distal coupler end of said coupler; wherein the handpiece comprises an optical element configured to receive said output light at the swivel coupler and transmit said output light to the distal handpiece end to generate the illumination beam at or near the working instrument and tooth surface.

67. The dental apparatus or method of any preceding or subsequent embodiment, wherein the optical element comprises a fiber optic element.

68. The dental apparatus or method of any preceding or subsequent embodiment; wherein the handpiece comprises an air-driven handpiece; wherein the coupler comprises a swivel coupler; and wherein the switching mechanism is located on the air-driven handpiece or the swivel coupler.

69. The dental apparatus or method of any preceding or subsequent embodiment; wherein the handpiece comprises an electric contra angle; wherein the coupler comprises a handpiece motor; and wherein the switching mechanism is located on the electrical handpiece or the handpiece motor.

70. The dental apparatus or method of any preceding or subsequent embodiment, further comprising: motor control box coupled to the handpiece motor for supplying electrical power to the handpiece motor; wherein the switching mechanism is located on or in the control box.

71. The dental apparatus or method of any preceding or subsequent embodiment: said handpiece comprising a mechanically driven contra angle; said motor comprising a centrally-located output shaft at said distal coupler end to drive the mechanically driven contra angle; wherein the handpiece optical element and illumination source output light are off-center with respect to the output shaft.

72. The dental apparatus or method of any preceding or subsequent embodiment, wherein the working instrument comprises a cutting tool.

73. The dental apparatus or method of any preceding or subsequent embodiment, wherein the working instrument comprises a scaler or other powered dental instrument for removing plaque, calculus or tartar from the tooth surface.

74. The dental apparatus or method of any preceding or subsequent embodiment, wherein the emitted 405 nm light is used to detect plaque, calculus or tartar from the tooth surface simultaneous with its removal.

75. The dental apparatus or method of any preceding or subsequent embodiment, wherein the white light is used to assist in occlusal adjustment and final surface finishing of said tooth surface.

76. The dental apparatus or method of any preceding or subsequent embodiment, comprising: directing a working end of the dental instrument at a surface of the tooth; emitting a 405 nm light from the working end of the dental instrument to detect and remove decay; switching a wavelength of the emitted light from 405 nm to white light; and directing the emitted white light at said tooth surface to assist in occlusal adjustment and final surface finishing of the tooth surface.

77. The dental apparatus or method of any preceding or subsequent embodiment, further comprising: applying restorative material to the tooth surface after said removal of decay; wherein the emitted 405 nm light is used to enhance visual differentiation between excess restorative material and tooth surface.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A dental apparatus, comprising:
(a) a housing configured for attachment of a dental instrument at an end of the housing, said dental instrument configured for performing a dental procedure on a tooth surface;
(b) a dual-wavelength lamp coupled to the housing and configured to provide an illumination beam for illuminating an area on or near the tooth surface;
(c) the dual-wavelength lamp comprising first and second adjacent illumination sources, wherein each said illumination source is configured to generate said illumination beam;
(d) wherein said first illumination source is configured to generate the illumination beam a first wavelength and wherein said second illumination source is configured to generate the illumination beam at a second wavelength;
(e) a switching mechanism connected to the first and second illumination sources and configured to selectively apply power to only one of said illumination sources at a time;
(f) an optical barrier disposed between the first and second illumination sources and configured to shield a non-powered illumination source from radiation emitted by an adjacent powered illumination source, wherein fluorescence of a non-powered illumination source is minimized or inhibited as a result of said barrier, thus minimizing or inhibiting emission of a non-selected wavelength along with a selected wavelength.

2. The apparatus of claim 1, wherein the switching mechanism is configured to switch polarity of power delivered to the dual-wavelength lamp thereby switching power between the first illumination source and the second illumination source to change the wavelength of the illumination beam.

3. The apparatus of claim 1, wherein the first and second wavelengths comprise white light and light at a wavelength of about 405 nm, respectively.

4. The apparatus of claim 3:
wherein the first illumination source comprises a first light emitting diode (LED) configured to emit light at the first wavelength; and
wherein the second illumination source comprises a second LED configured to emit light at the second wavelength.

5. The apparatus of claim 4, wherein each said LED is oriented to generate an associated illumination beam at substantially a same focal point.

6. The apparatus of claim 4, wherein the first and second LEDs are configured to emit light at a sufficient output density and intensity to travel through a light guide of a diameter not to exceed about 0.125 inches.

7. The apparatus of claim 1:
wherein the dental instrument comprises a dental handpiece having a proximal handpiece end, and a distal handpiece end for attaching a working instrument;
wherein the housing comprises a coupler having a proximal coupler end for releasably coupling to a supply hose and a distal coupler end for releasably connecting to the proximal handpiece end of the dental handpiece;
wherein the dual-wavelength lamp is configured to output light at the first and second wavelengths at said distal coupler end of said coupler;
wherein the dental handpiece comprises an optical element configured to receive output light from the coupler and transmit said output light to the distal handpiece end to generate the illumination beam at or near the working instrument and tooth surface.

8. The apparatus of claim 10, wherein the optical element comprises a fiber optic element.

9. The apparatus of claim 10:
wherein the handpiece comprises an air-driven handpiece;
wherein the coupler comprises a swivel coupler; and
wherein the switching mechanism is located on the air-driven handpiece or the swivel coupler.

10. The dental apparatus of claim 10:
wherein the handpiece comprises an electric contra angle;
wherein the coupler comprises a handpiece motor; and
wherein the switching mechanism is located on the electrical handpiece or the handpiece motor.

11. The apparatus of claim 13, further comprising:
motor control box coupled to the handpiece motor for supplying electrical power to the handpiece motor;
wherein the switching mechanism is located on or in the control box.

12. The apparatus of claim 13:
said handpiece comprising a mechanically driven contra angle;
said motor comprising a centrally-located output shaft at said distal coupler end to drive the mechanically driven contra angle;
wherein the handpiece optical element and illumination source output light are off-center with respect to the output shaft.

13. The dental apparatus of claim 1, wherein the dental instrument comprises a cutting tool, a scaler, or other powered dental instrument for removing plaque, calculus or tartar from the tooth surface.

14. A dental apparatus, comprising:
(a) a coupler having a proximal coupler end for releasably coupling to a supply hose and a distal coupler end for releasably connecting to a proximal end of a dental instrument configured for performing a dental procedure on a tooth surface;
(b) a dual-wavelength lamp disposed in the coupler and configured to provide an illumination beam for illuminating an area on or near the tooth surface;
(c) wherein said dual-wavelength lamp is configured to output light at the first and second wavelengths at said distal coupler end of said coupler;
(d) the dual-dual wavelength lamp comprising first and second adjacent illumination sources, wherein each said illumination source is configured to generate said illumination beam;
(e) wherein said first illumination source is configured to generate the illumination beam a first wavelength and wherein said second illumination source is configured to generate the illumination beam at a second wavelength;
(f) a switching mechanism connected to the first and second illumination sources and configured to selectively apply power to only one of said illumination sources at a time;
(g) an optical barrier disposed between the first and second illumination sources and configured to shield a non-powered illumination source from radiation emitted by an adjacent powered illumination source, wherein fluorescence of a non-powered illumination source is minimized or inhibited as a result of said barrier, thus minimizing or inhibiting emission of a non-selected wavelength along with a selected wavelength.

15. The apparatus of claim 14, wherein the switching mechanism is configured to switch polarity of power delivered to the dual-wavelength lamp thereby switching power between the first illumination source and the second illumination source to change the wavelength of the illumination beam.

16. The apparatus of claim 140, wherein the first and second wavelengths comprise white light and light at a wavelength of about 405 nm, respectively.

17. The apparatus of claim 16:
wherein the first illumination source comprises a first light emitting diode (LED) configured to emit light at the first wavelength; and
wherein the second illumination source comprises a second LED configured to emit light at the second wavelength.

18. The apparatus of claim 17, wherein each said LED is oriented to generate an associated illumination beam at substantially a same focal point.

19. The apparatus of claim 17, wherein the first and second LEDs are configured to emit light at a sufficient output density and intensity to travel through a light guide of a diameter not to exceed about 0.125 inches.

20. The apparatus of claim 17:
wherein the optical barrier is configured to shield a non-powered LED from radiation emitted by an adjacent powered LED; and
wherein fluorescence of the non-powered LED is minimized or inhibited as a result of said barrier, thus minimizing or inhibiting emission of a non-selected wavelength along with a selected wavelength.

21. The apparatus of claim 14:
wherein the dental instrument comprises a dental handpiece having a proximal handpiece end, and a distal handpiece end for attaching a working instrument;
wherein the dental handpiece comprises an optical element configured to receive output light from the coupler and transmit said output light to the distal handpiece end to generate the illumination beam at or near the working instrument and tooth surface.

22. The apparatus of claim 21, wherein the optical element comprises a fiber optic element.

23. The apparatus of claim 21:
wherein the handpiece comprises an air-driven handpiece;
wherein the coupler comprises a swivel coupler; and
wherein the switching mechanism is located on the air-driven handpiece or the swivel coupler.

24. The dental apparatus of claim 21:
wherein the handpiece comprises an electric contra angle;
wherein the coupler comprises a handpiece motor; and
wherein the switching mechanism is located on the electrical handpiece or the handpiece motor.

25. The apparatus of claim 24, further comprising:
motor control box coupled to the handpiece motor for supplying electrical power to the handpiece motor;
wherein the switching mechanism is located on or in the control box.

26. The apparatus of claim 24:
said handpiece comprising a mechanically driven contra angle;
said motor comprising a centrally-located output shaft at said distal coupler end to drive the mechanically driven contra angle;
wherein the handpiece optical element and illumination source output light are off-center with respect to the output shaft.

27. The dental apparatus of claim 14, wherein the dental instrument comprises a cutting tool, a scaler, or other powered dental instrument for removing plaque, calculus or tartar from the tooth surface.
